SEQUENCE LISTING (63 sequences)

SEQ ID NO: 1 - protein, 183 aa, Artificial Sequence
  The sequene is synthesized.
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 458 aa, Artificial Sequence
  The sequence is synthesized.
QVKLQQSGPGILQPSQTLSLTCSFSGFSLSTSGMGVGWIRQPSGKGLEWLAHIWWDDDKRYNSALKSRLTISKDTSSNQVFLKIASVDSADTATYYCARIGGLYVDYLMDYWGQGTSVTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 3 - protein, 428 aa, Artificial Sequence
  The sequence is synthesized.
DILMTQSPATLSVTPGDRVSLSCRASQSISDYLHWYQQKSHESPRLLIKYASQSISGIPSRFSGSGSGSDFTLSIDSVEPEDVGVYYCQNGHSFPWTFGGGTKLEIRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECDILMTQSPATLSVTPGDRVSLSCRASQSISDYLHWYQQKSHESPRLLIKYASQSISGIPSRFSGSGSGSDFTLSIDSVEPEDVGVYYCQNGHSFPWTFGGGTKLEIRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 122 aa, Artificial Sequence
  The sequence is synthesized.
QVKLQQSGPGILQPSQTLSLTCSFSGFSLSTSGMGVGWIRQPSGKGLEWLAHIWWDDDKRYNSALKSRLTISKDTSSNQVFLKIASVDSADTATYYCARIGGLYVDYLMDYWGQGTSVTVSS

SEQ ID NO: 5 - protein, 107 aa, Artificial Sequence
  The sequence is synthesized.
DILMTQSPATLSVTPGDRVSLSCRASQSISDYLHWYQQKSHESPRLLIKYASQSISGIPSRFSGSGSGSDFTLSIDSVEPEDVGVYYCQNGHSFPWTFGGGTKLEIR

SEQ ID NO: 6 - protein, 10 aa, Artificial Sequence
  The sequence is synthesized.
GFSLSTSGMG

SEQ ID NO: 7 - protein, 5 aa, Artificial Sequence
  The sequence is synthesized.
WWDDD

SEQ ID NO: 8 - protein, 14 aa, Artificial Sequence
  The sequence is synthesized.
ARIGGLYVDYLMDY

SEQ ID NO: 9 - protein, 6 aa, Artificial Sequence
  The sequence is synthesized.
QSISDY

SEQ ID NO: 10 - protein, 3 aa, Artificial Sequence
  The sequence is synthesized.
YAS

SEQ ID NO: 11 - protein, 9 aa, Artificial Sequence
  The sequence is synthesized.
QNGHSFPWT

SEQ ID NO: 12 - protein, 25 aa, Artificial Sequence
  The sequence is synthesized.
QVKLQQSGPGILQPSQTLSLTCSFS

SEQ ID NO: 13 - protein, 18 aa, Artificial Sequence
  The sequence is synthesized.
VGWIRQPSGKGLEWLAHI

SEQ ID NO: 14 - protein, 39 aa, Artificial Sequence
  The sequence is synthesized.
KRYNSALKSRLTISKDTSSNQVFLKIASVDSADTATYYC

SEQ ID NO: 15 - protein, 11 aa, Artificial Sequence
  The sequence is synthesized.
WGQGTSVTVSS

SEQ ID NO: 16 - protein, 26 aa, Artificial Sequence
  The sequence is synthesized.
DILMTQSPATLSVTPGDRVSLSCRAS

SEQ ID NO: 17 - protein, 17 aa, Artificial Sequence
  The sequence is synthesized.
LHWYQQKSHESPRLLIK

SEQ ID NO: 18 - protein, 36 aa, Artificial Sequence
  The sequence is synthesized.
QSISGIPSRFSGSGSGSDFTLSIDSVEPEDVGVYYC

SEQ ID NO: 19 - protein, 10 aa, Artificial Sequence
  The sequence is synthesized.
FGGGTKLEIR

SEQ ID NO: 20 - DNA, 1374 bp, Artificial Sequence
  The sequence is synthesized.
caggtcaaactgcagcagtctggccctgggatattgcagccctcccagaccctcagtctgacttgttctttctctgggttttcactgagcacttctggtatgggagtaggctggattcgtcagccttcagggaagggtctggagtggctggcacacatttggtgggatgacgacaagcgctataattcagccctgaagagccgactgacaatctccaaggatacctccagcaaccaggtattcctcaagatcgccagtgtggacagtgcagatactgccacatattactgcgctcgaattggtggcctctacgttgactatcttatggactactggggtcaaggaacctcagtcaccgtctcctcagccaaaacaacacccccatcagtctatccactggcccctgggtgtggagatacaactggttcctccgtgacctctgggtgcctggtcaaggggtacttccctgagccagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtgaacaacaaagacctcccatcacccatcgagagaaccatctcaaaaattaaagggctagtcagagctccacaagtatacactttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagttcttgactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 21 - DNA, 642 bp, Artificial Sequence
  The sequence is synthesized.
gacattctgatgacccagtctccagccaccctgtctgtgactccaggagatagggtctctctttcctgcagggccagtcagagtattagcgactacttacactggtatcaacaaaaatcacatgagtctccaaggcttctcatcaagtatgcttcccaatccatctctgggatcccctccaggttcagtggcagtggatcagggtcagatttcactctcagtatcgacagtgtggaacctgaagatgttggagtgtattactgtcaaaatggtcacagttttccgtggacgttcggtggaggcaccaagctggaaatcaggcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 22 - DNA, 366 bp, Artificial Sequence
  The sequence is synthesized.
caggtcaaactgcagcagtctggccctgggatattgcagccctcccagaccctcagtctgacttgttctttctctgggttttcactgagcacttctggtatgggagtaggctggattcgtcagccttcagggaagggtctggagtggctggcacacatttggtgggatgacgacaagcgctataattcagccctgaagagccgactgacaatctccaaggatacctccagcaaccaggtattcctcaagatcgccagtgtggacagtgcagatactgccacatattactgcgctcgaattggtggcctctacgttgactatcttatggactactggggtcaaggaacctcagtcaccgtctcctca

SEQ ID NO: 23 - DNA, 321 bp, Artificial Sequence
  The sequence is synthesized.
gacattctgatgacccagtctccagccaccctgtctgtgactccaggagatagggtctctctttcctgcagggccagtcagagtattagcgactacttacactggtatcaacaaaaatcacatgagtctccaaggcttctcatcaagtatgcttcccaatccatctctgggatcccctccaggttcagtggcagtggatcagggtcagatttcactctcagtatcgacagtgtggaacctgaagatgttggagtgtattactgtcaaaatggtcacagttttccgtggacgttcggtggaggcaccaagctggaaatcagg

SEQ ID NO: 24 - protein, 214 aa, Artificial Sequence
  The sequence is synthesized.
EIVLTQSPDFQSVTPKEKVTITCRASQSISDYLHWYQQKPDQSPKLLIKYASQSISGVPSRFSGSGSGTDFTLTINSLEAEDAATYYCQNGHSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 25 - DNA, 642 bp, Artificial Sequence
  The sequence is synthesized.
gagatcgtgctgacccagagccccgacttccagagcgtgacccccaaggagaaggtgaccatcacctgcagggccagccaaagcatcagcgactacctgcactggtaccagcagaagcccgaccagagccccaagctgctgatcaagtacgcctcccagagcatcagcggcgtgccttccaggtttagcggcagcggcagcggcacagacttcaccctgaccatcaacagcctggaggccgaggacgccgctacctactactgccagaacggccacagcttcccctggaccttcggccagggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 26 - protein, 107 aa, Artificial Sequence
  The sequence is synthesized.
EIVLTQSPDFQSVTPKEKVTITCRASQSISDYLHWYQQKPDQSPKLLIKYASQSISGVPSRFSGSGSGTDFTLTINSLEAEDAATYYCQNGHSFPWTFGQGTKVEIK

SEQ ID NO: 27 - DNA, 321 bp, Artificial Sequence
  The sequence is synthesized.
gagatcgtgctgacccagagccccgacttccagagcgtgacccccaaggagaaggtgaccatcacctgcagggccagccaaagcatcagcgactacctgcactggtaccagcagaagcccgaccagagccccaagctgctgatcaagtacgcctcccagagcatcagcggcgtgccttccaggtttagcggcagcggcagcggcacagacttcaccctgaccatcaacagcctggaggccgaggacgccgctacctactactgccagaacggccacagcttcccctggaccttcggccagggcaccaaggtggagatcaag

SEQ ID NO: 28 - protein, 26 aa, Artificial Sequence
  The sequence is synthesized.
EIVLTQSPDFQSVTPKEKVTITCRAS

SEQ ID NO: 29 - protein, 17 aa, Artificial Sequence
  The sequence is synthesized.
LHWYQQKPDQSPKLLIK

SEQ ID NO: 30 - protein, 36 aa, Artificial Sequence
  The sequence is synthesized.
QSISGVPSRFSGSGSGTDFTLTINSLEAEDAATYYC

SEQ ID NO: 31 - protein, 10 aa, Artificial Sequence
  The sequence is synthesized.
FGQGTKVEIK

SEQ ID NO: 32 - protein, 214 aa, Artificial Sequence
  The sequence is synthesized.
EIVLTQSPATLSLSPGERATLSCRASQSISDYLHWYQQKPGQAPRLLIYYASQSISGIPARFSGSGSGTDFTLTISSLEPEDFAVYYCQNGHSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 33 - DNA, 642 bp, Artificial Sequence
  The sequence is synthesized.
gagatcgtgctgacccagagccctgccacactgagcctgagccctggcgaaagggccaccctgagctgcagagccagccaaagcatcagcgactacctgcactggtaccagcagaagcccggccaggcccctagactgctgatctactacgccagccagagcatcagcggcatccctgccagatttagcggcagcggcagcggcacagactttaccctgaccatcagcagcctggagcccgaggacttcgccgtgtactactgccagaatggccacagcttcccctggaccttcggccagggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 34 - protein, 107 aa, Artificial Sequence
  The sequence is synthesized.
EIVLTQSPATLSLSPGERATLSCRASQSISDYLHWYQQKPGQAPRLLIYYASQSISGIPARFSGSGSGTDFTLTISSLEPEDFAVYYCQNGHSFPWTFGQGTKVEIK

SEQ ID NO: 35 - DNA, 321 bp, Artificial Sequence
  The sequence is synthesized.
gagatcgtgctgacccagagccctgccacactgagcctgagccctggcgaaagggccaccctgagctgcagagccagccaaagcatcagcgactacctgcactggtaccagcagaagcccggccaggcccctagactgctgatctactacgccagccagagcatcagcggcatccctgccagatttagcggcagcggcagcggcacagactttaccctgaccatcagcagcctggagcccgaggacttcgccgtgtactactgccagaatggccacagcttcccctggaccttcggccagggcaccaaggtggagatcaag

SEQ ID NO: 36 - protein, 26 aa, Artificial Sequence [sequence omitted]

SEQ ID NO: 37 - protein, 17 aa, Artificial Sequence
  The sequence is synthesized.
LHWYQQKPGQAPRLLIY

SEQ ID NO: 38 - protein, 36 aa, Artificial Sequence
  The sequence is synthesized.
QSISGIPARFSGSGSGTDFTLTISSLEPEDFAVYYC

SEQ ID NO: 39 - protein, 10 aa, Artificial Sequence
  The sequence is synthesized.
FGQGTKVEIK

SEQ ID NO: 40 - protein, 214 aa, Artificial Sequence
  The sequence is synthesized.
DIQMTQSPSSLSASVGDRVTITCRASQSISDYLHWYQQKPGKAPKLLIYYASQSISGVPSRFSGSGSGTDFTLTISSLQPEDFATYYCQNGHSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 41 - DNA, 642 bp, Artificial Sequence
  The sequence is synthesized.
gacatccagatgacccagagccctagcagcctgagcgccagcgtgggcgatagggtgaccatcacctgcagggccagccaaagcatcagcgactacctgcactggtaccagcagaagcccggcaaggcccccaagctgctgatctactacgccagccagagcatcagcggcgtgcctagcagattcagcggctccggctccggcaccgactttaccctgaccatcagctccctgcagcccgaggacttcgccacctactactgccagaacggccacagcttcccctggaccttcggccagggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 42 - protein, 107 aa, Artificial Sequence
  The sequence is synthesized.
DIQMTQSPSSLSASVGDRVTITCRASQSISDYLHWYQQKPGKAPKLLIYYASQSISGVPSRFSGSGSGTDFTLTISSLQPEDFATYYCQNGHSFPWTFGQGTKVEIK

SEQ ID NO: 43 - DNA, 321 bp, Artificial Sequence
  The sequence is synthesized.
gacatccagatgacccagagccctagcagcctgagcgccagcgtgggcgatagggtgaccatcacctgcagggccagccaaagcatcagcgactacctgcactggtaccagcagaagcccggcaaggcccccaagctgctgatctactacgccagccagagcatcagcggcgtgcctagcagattcagcggctccggctccggcaccgactttaccctgaccatcagctccctgcagcccgaggacttcgccacctactactgccagaacggccacagcttcccctggaccttcggccagggcaccaaggtggagatcaag

SEQ ID NO: 44 - protein, 26 aa, Artificial Sequence [sequence omitted]

SEQ ID NO: 45 - protein, 17 aa, Artificial Sequence
  The sequence is synthesized.
LHWYQQKPGKAPKLLIY

SEQ ID NO: 46 - protein, 36 aa, Artificial Sequence
  The sequence is synthesized.
QSISGVPSRFSGSGSGTDFTLTISSLQPEDFATYYC

SEQ ID NO: 47 - protein, 10 aa, Artificial Sequence
  The sequence is synthesized.
FGQGTKVEIK

SEQ ID NO: 48 - protein, 452 aa, Artificial Sequence
  The sequence is synthesized.
QVTLKESGPALVKPTQTLTLTCTFSGFSLSTSGMGVGWIRQPPGKALEWLAHIWWDDDKRYNSALKSRLTISKDTSKNQVVLTMTNMDPVDTATYYCARIGGLYVDYLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 49 - DNA, 1356 bp, Artificial Sequence
  The sequence is synthesized.
caggtgacactgaaggagagcggccccgccctggtgaaacctacccagaccctgaccctgacctgcaccttcagcggcttcagcctgagcacaagcggcatgggcgtgggctggatcagacagcctcctggcaaggccctggagtggctggcccacatctggtgggacgacgacaagaggtacaacagcgccctgaagagcaggctgaccatcagcaaggacaccagcaagaaccaggtggtgctgaccatgaccaacatggaccccgtggacaccgccacctactactgcgccagaatcggcggcctgtacgtggactacctgatggactactggggccagggcaccctggtgacagtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaa

SEQ ID NO: 50 - protein, 122 aa, Artificial Sequence
  The sequence is synthesized.
QVTLKESGPALVKPTQTLTLTCTFSGFSLSTSGMGVGWIRQPPGKALEWLAHIWWDDDKRYNSALKSRLTISKDTSKNQVVLTMTNMDPVDTATYYCARIGGLYVDYLMDYWGQGTLVTVSS

SEQ ID NO: 51 - DNA, 366 bp, Artificial Sequence
  The sequence is synthesized.
caggtgacactgaaggagagcggccccgccctggtgaaacctacccagaccctgaccctgacctgcaccttcagcggcttcagcctgagcacaagcggcatgggcgtgggctggatcagacagcctcctggcaaggccctggagtggctggcccacatctggtgggacgacgacaagaggtacaacagcgccctgaagagcaggctgaccatcagcaaggacaccagcaagaaccaggtggtgctgaccatgaccaacatggaccccgtggacaccgccacctactactgcgccagaatcggcggcctgtacgtggactacctgatggactactggggccagggcaccctggtgacagtgagcagc

SEQ ID NO: 52 - protein, 25 aa, Artificial Sequence
  The sequence is synthesized.
QVTLKESGPALVKPTQTLTLTCTFS

SEQ ID NO: 53 - protein, 18 aa, Artificial Sequence
  The sequence is synthesized.
VGWIRQPPGKALEWLAHI

SEQ ID NO: 54 - protein, 39 aa, Artificial Sequence
  The sequence is synthesized.
KRYNSALKSRLTISKDTSKNQVVLTMTNMDPVDTATYYC

SEQ ID NO: 55 - protein, 11 aa, Artificial Sequence
  The sequence is synthesized.
WGQGTLVTVSS

SEQ ID NO: 56 - protein, 452 aa, Artificial Sequence
  The sequence is synthesized.
QVTLKESGPVLVKPTETLTLTCTVSGFSLSTSGMGVGWIRQPPGKALEWLAHIWWDDDKRYNSALKSRLTISKDTSKSQVVLTMTNMDPVDTATYYCARIGGLYVDYLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 57 - DNA, 1356 bp, Artificial Sequence
  The sequence is synthesized.
caggtgacactgaaggagagcggccccgtgctggtgaaacccaccgagaccctgaccctgacctgcaccgtgagcggattcagcctgagcacaagcggcatgggcgtgggctggattaggcagccccctggcaaggctctggagtggctggcccacatctggtgggacgacgacaagaggtacaacagcgccctgaagagcaggctcaccatctccaaggacaccagcaagagccaggtggtgctgaccatgaccaacatggaccccgtggacaccgccacctactactgcgccaggatcggcggcctgtacgtggactacctgatggactactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaa

SEQ ID NO: 58 - protein, 122 aa, Artificial Sequence
  The sequence is synthesized.
QVTLKESGPVLVKPTETLTLTCTVSGFSLSTSGMGVGWIRQPPGKALEWLAHIWWDDDKRYNSALKSRLTISKDTSKSQVVLTMTNMDPVDTATYYCARIGGLYVDYLMDYWGQGTLVTVSS

SEQ ID NO: 59 - DNA, 366 bp, Artificial Sequence
  The sequence is synthesized.
caggtgacactgaaggagagcggccccgtgctggtgaaacccaccgagaccctgaccctgacctgcaccgtgagcggattcagcctgagcacaagcggcatgggcgtgggctggattaggcagccccctggcaaggctctggagtggctggcccacatctggtgggacgacgacaagaggtacaacagcgccctgaagagcaggctcaccatctccaaggacaccagcaagagccaggtggtgctgaccatgaccaacatggaccccgtggacaccgccacctactactgcgccaggatcggcggcctgtacgtggactacctgatggactactggggccagggcacactggtgaccgtgagcagc

SEQ ID NO: 60 - protein, 25 aa, Artificial Sequence
  The sequence is synthesized.
QVTLKESGPVLVKPTETLTLTCTVS

SEQ ID NO: 61 - protein, 18 aa, Artificial Sequence
  The sequence is synthesized.
VGWIRQPPGKALEWLAHI

SEQ ID NO: 62 - protein, 39 aa, Artificial Sequence
  The sequence is synthesized.
KRYNSALKSRLTISKDTSKSQVVLTMTNMDPVDTATYYC

SEQ ID NO: 63 - protein, 11 aa, Artificial Sequence
  The sequence is synthesized.
WGQGTLVTVSS

(19) United States
(12) Patent Application Publication
(10) Pub. No.: US 2021/0395357 A1
(43) Pub. Date: Dec. 23, 2021

(54) HUMANIZED MONOCLONAL ANTIBODIES AGAINST INTERLEUKIN-6, ENCODING GENES AND USES THEREOF

(71) Applicant: Iphase Pharma Service LLC, North Wales, PA (US)

(72) Inventors: Hongjun LIU, Beijing (CN); Li LI, Beijing (CN)

(21) Appl. No.: 17/283,842

(22) PCT Filed: Sep. 27, 2019
(86) PCT No.: PCT/CN2019/108733

§ 371 Date: Apr. 8, 2021

(30) Foreign Application Priority Data
Oct. 10, 2018 (CN) 201811181118.3

(51) Int. Cl. C07K 16/24

(52) U.S. Cl. CPC C07K 16/248; C07K 2317/24; C07K 2317/31; C07K 2317/33; C07K 2319/20; C07K 2317/565; C07K 2317/567; C07K 2317/76; C07K 2317/92; C07K 2317/35

(57) ABSTRACT

The present invention relates to an anti-IL-6 antibody, a pharmaceutical composition or a kit comprising the same, and uses thereof.

### FIELD OF THE INVENTION

The present invention relates to antibodies, specially relates to humanized monoclonal antibody against interleukin-6 (IL-6), encoding gene thereof, and use thereof.

### BACKGROUND OF THE INVENTION

Interleukin-6 (IL-6) (also known as interferon-β2, B cell differentiation factor, B cell stimulating factor-2, hepatocyte stimulating factor, hybridoma growth factor) is a multifunctional cytokine produced by many different cell types and involved in many biological processes, including regulation of acute inflammatory response, regulation of specific immune response (including B cell and T cell differentiation), bone metabolism, platelet production, epidermal cell proliferation, menstruation, neural cell differentiation, neuroprotection, aging, cancer and inflammation in Alzheimer's disease. See A. Papassotiropoulos et al. (2001), Neurobiology of Aging, 22: 863-871.

The gene encoding human IL-6 contains five exons and four introns, and is located on the short arm 7p21 of chromosome 7. The translation and post-translational modification of IL-6 RNA produces proteins of 21 kDa to 28 kDa with 184 amino acids. See A. Papassotiropoulos et al. (2001), Neurobiology of Aging, 22:863-871.

IL-6 can bind to IL-6 receptor complexes expressed on mitogen-activated B cells, T cells, peripheral monocytes, and certain tumor cells. The receptor complex consists of at least one subunit of the signal transduction glycoprotein gp130 and the IL-6 receptor (“IL-6R”) (also known as gp80). IL-6R can also exist in a soluble form (“sIL-6R”). IL-6 binds to IL-6R and then dimerizes the signal transduction receptor gp130. See Jones, S A, J. Immunology, 175: 3463 3468 (2005). The cytokine families including IL-6, LIF, oncostatin M, IL-11, CNTF, and CT-1 all signal via gp130 after binding to their associated receptors, wherein the intracellular segment of gp130 contains conserved sequences related to the activation of tyrosine kinases. IL-6 activates a variety of intracellular kinase molecules and transcription factors through the interaction with its receptor complex and ultimately activates the expression of related genes.

IL-6 is a pleiotropic pro-inflammatory cytokine that can regulate the acute phase response and the transition from innate to adaptive immune response. IL-6 promotes the synthesis of reactive proteins involved in the acute phase in the liver, leading to symptoms such as fever, chills, and fatigue. It stimulates B cell differentiation and antibody secretion, and prevents apoptosis of activated B cells. IL-6 activates and induces T cell proliferation, and in the presence of IL-2, induces the differentiation of mature and immature CD8 T cells into cytotoxic T cells. IL-6 is also involved in the differentiation of Th17 cells and the production of IL-17, and inhibits the differentiation of regulatory T cells (Treg). IL-6 also activates osteoclasts, synovial cells, neutrophils and other hematopoietic cells. See Park, et al. (2007), Bulletin of the NYU Hospital for Joint Diseases 65 (suppl 1): S4-10; Guerne, et al. (1989), J Clin Invest., 83(2): 585-92; Houssiau, et al. (1988), Arthritis Rheum., 31(6): 784-8; Nishimotor, et al. (2006), Nat Clin Pract Rheumatol., 2(11): 619-26; Kishimoto (1989), Blood, 74(1): 1-10; Van Snick (1990), Annu Rev Immunol., 8: 253-78.

The function of IL-6 is not limited to immune response. It plays a role in hematopoiesis, platelet production, osteoclast formation, and induction of acute hepatic response, leading to the increase of C-reactive protein (CRP) and serum amyloid A (SAA) protein. It is also a growth factor for epidermal keratinocytes, glomerular mesangial cells, myeloma and plasmacytoma cells. See Grossman, et al. (1989), Prot Natl Acad Sci., 86(16): 6367-6371; Horii, et al. (1989), J Immunol., 143(12): 3949-3955; Kawano, et al. (1988), Nature, 332: 83-85. In the body, stimulated monocytes, fibroblasts, and endothelial cells are the main sources of IL-6. Other cells such as macrophages, T and B lymphocytes, granulocytes, keratinocytes, mast cells, osteoblasts, chondrocytes, glial cells, and smooth muscle cells also produce IL-6 after stimulation (Kishimoto, T., Blood 74: 1-10 (1989) and Kurihara, N. et al., J. Immunology 144: 4226-4230 (1990)). Several tumor cells also produce IL-6, and IL-6 has been shown to be a prognostic factor for prostate cancer progression. Except for tumor cells that constitutively produce IL-6, normal cells do not express IL-6 unless they are properly stimulated. IL-6 production can be regulated by IL-6 itself, and depending on the cell type, IL-6 can stimulate or inhibit its own synthesis.

Elevated levels of IL-6 have been observed in many types of cancers, including breast cancer, leukemia, ovarian cancer, prostate cancer, pancreatic cancer, lymphoma, lung cancer, renal cell carcinoma, colorectal cancer, and multiple myeloma. See Chopra, et al. (2004), MJAFI, 60:45-49; Songur, et al. (2004), Tumori, 90:196-200; Blay, et al. (1992), Cancer Research, 52: 3317-3322; Nikiteas, et al. (2005), World J. Gasterenterol., 11:1639-1643; Heikkila, et al. (2008), Eur J Cancer, 44:937-945. Clinical studies (Trikha, et al (2003), Clinical Cancer Research 9: 4653-4665) showed that the subjects' condition was improved after the administration of various anti-IL-6 antibodies. In cancer cases where IL-6 promotes the proliferation or survival of cancer cells, the role of antibodies is more obvious.

IL-6 is believed to play a role in the development of many diseases and conditions, including but not limited to fatigue, cachexia, inflammatory diseases, autoimmune diseases, skeletal system diseases, fever, cancer, heart disease, obesity, diabetes, asthma, Alzheimer's disease, multicentric Castleman disease, multiple sclerosis and rheumatoid arthritis. See, for example, WO2011/066374, WO2011/066371, WO2011/066378 and WO2011/0666369.

In addition to its direct role in the pathogenesis of certain cancers and other diseases, long-term elevated IL-6 levels seem to have an adverse effect on the health and quality of life of patients. Elevated IL-6 levels are associated with cachexia and fever, and can reduce serum albumin. Gauldie, et al. (1987), PNAS, 84: 7251-7253; Heinric, et al. (1990), Biochem J., 265(3): 621-636; Zamir, et al. (1993), Metabolism, 42: 204-208; Zamir, et al. (1992), Arch Surg, 127: 170-174. Inhibition of IL-6 by neutralizing antibodies can improve fever and cachexia in cancer patients, but it has not been reported to improve serum albumin levels. Emille, et al. (1994), Blood, 84: 2472-2479; Blay, et al. (1992), Cancer Research, 52: 3317-3322; Bataille, et al. (1995), Blood, 86: 685-691.

In addition, Siltuximab, an IL-6 monoclonal antibody for the treatment of multicentric Castleman disease, was approved for marketing in the United States in 2014. This is the only anti-IL-6 monoclonal antibody currently on the market worldwide. The monoclonal antibodies Sirukumab and Olokizumab used in the treatment of rheumatoid arthritis are also expected to be approved for marketing in the near future, see Kim G W et al. (2015), Arch Pharm Res., 38(5):575-84. There is no monoclonal antibody against IL-6 target on the market in China.

### SUMMARY OF THE INVENTION

In one aspect of the present disclosure, it relates to antibodies or antigen-binding fragments thereof, in particular, the antibodies or antigen-binding fragments thereof bind to IL6, preferably human IL6, wherein: (1) the antibody comprises:

HCDR1, which comprises or consists of a sequence as shown in SEQ ID NO: 6, a sequence having at least 90%, preferably at least 91%, 92%, 93%, 94%, 95%, 96%, 97%, 98%, 99% or 100% sequence identity with SEQ ID NO: 6, or an amino acid sequence with one or more (preferably 2 or 3) conservative amino acid mutations (preferably substitutions, insertions or deletions) compared to SEQ ID NO: 6,

HCDR2, which comprises or consists of a sequence as shown in SEQ ID NO: 7, a sequence having at least 90%, preferably at least 91%, 92%, 93%, 94%, 95%, 96%, 97%, 98%, 99% or 100% sequence identity with SEQ ID NO: 7, or an amino acid sequence with one or more (preferably 2 or 3) conservative amino acid mutations (preferably substitutions, insertions or deletions) compared to SEQ ID NO: 7, and

HCDR3, which comprises or consists of a sequence as shown in SEQ ID NO: 8, a sequence having at least 90%, preferably at least 91%, 92%, 93%, 94%, 95%, 96%, 97%, 98%, 99% or 100% sequence identity with SEQ ID NO: 8, or an amino acid sequence with one or more (preferably 2 or 3) conservative amino acid mutations (preferably substitutions, insertions or deletions) compared to SEQ ID NO: 8,

And the antibody also includes:

LCDR1, which comprises or consists of an amino acid sequence as shown in SEQ ID NO: 9, a sequence having at least 90%, preferably at least 91%, 92%, 93%, 94%, 95%, 96%, 97%, 98%, 99% or 100% sequence identity with SEQ ID NO: 9, or an amino acid sequence with one or more (preferably 2 or 3) conservative amino acid mutations (preferably substitutions, insertions or deletions) compared to SEQ ID NO: 9,

LCDR2, which comprises or consists of an amino acid sequence as shown in SEQ ID NO: 10, a sequence having at least 90%, preferably at least 91%, 92%, 93%, 94%, 95%, 96%, 97%, 98%, 99% or 100% sequence identity with SEQ ID NO: 10, or an amino acid sequence with one or more (preferably 2 or 3) conservative amino acid mutations (preferably substitutions, insertions or deletions) compared to SEQ ID NO: 10, and

LCDR3, which comprises or consists of a sequence as shown in SEQ ID NO: 11, a sequence having at least 90%, preferably at least 91%, 92%, 93%, 94%, 95%, 96%, 97%, 98%, 99% or 100% sequence identity with SEQ ID NO: 11, or an amino acid sequence with one or more (preferably 2 or 3) conservative amino acid mutations (preferably substitutions, insertions or deletions) compared to SEQ ID NO: 11.

In a specific embodiment, the antibody includes:

(1) (i) a heavy chain variable region, which comprises or consists of the following sequence:

an amino acid sequence as shown in SEQ ID NO: 4, or

a sequence having at least 90%, preferably at least 91%, 92%, 93%, 94%, 95%, 96%, 97%, 98%, 99% or 100% sequence identity with SEQ ID NO: 4, or

an amino acid sequence with one or more (preferably 1, 2, 3, 4, 5, 6, 7, 8, 9 or 10) conservative amino acid mutations (preferably substitutions, insertions or deletions) compared to SEQ ID NO: 4, and

(ii) a light chain variable region, which comprises or consists of the following sequence:

an amino acid sequence as shown in SEQ ID NO: 5, or

a sequence having at least 90%, preferably at least 91%, 92%, 93%, 94%, 95%, 96%, 97%, 98%, 99% or 100% sequence identity with SEQ ID NO: 5, or

an amino acid sequence with one or (preferably 1, 2, 3, 4, 5, 6, 7, 8, 9 or 10) conservative amino acid mutations (preferably substitutions, insertions or deletions) compared to SEQ ID NO: 5;

(2) (i) a heavy chain variable region, which comprises or consists of the following sequence:

an amino acid sequence as shown in SEQ ID NO: 50, or

a sequence having at least 90%, preferably at least 91%, 92%, 93%, 94%, 95%, 96%, 97%, 98%, 99% or 100% sequence identity with SEQ ID NO: 50, or

an amino acid sequence with one or (preferably 1, 2, 3, 4, 5, 6, 7, 8, 9 or 10) conservative amino acid mutations (preferably substitutions, insertions or deletions) compared to SEQ ID NO: 50, and

(ii) a light chain variable region, which comprises or consists of the following sequence:

an amino acid sequence as shown in SEQ ID NO: 26, or

a sequence having at least 90%, preferably at least 91%, 92%, 93%, 94%, 95%, 96%, 97%, 98%, 99% or 100% sequence identity with SEQ ID NO: 26, or

an amino acid sequence with one or (preferably 1, 2, 3, 4, 5, 6, 7, 8, 9 or 10) conservative amino acid mutations (preferably substitutions, insertions or deletions) compared to SEQ ID NO: 26;

(3) (i) a heavy chain variable region, which comprises or consists of the following sequence:

an amino acid sequence as shown in SEQ ID NO: 58, or

a sequence having at least 90%, preferably at least 91%, 92%, 93%, 94%, 95%, 96%, 97%, 98%, 99% or 100% sequence identity with SEQ ID NO: 58, or

an amino acid sequence with one or (preferably 1, 2, 3, 4, 5, 6, 7, 8, 9 or 10) conservative amino acid mutations (preferably substitutions, insertions or deletions) compared to SEQ ID NO: 58, and

(ii) a light chain variable region, which comprises or consists of the following sequence:

an amino acid sequence as shown in SEQ ID NO: 26, or

a sequence having at least 90%, preferably at least 91%, 92%, 93%, 94%, 95%, 96%, 97%, 98%, 99% or 100% sequence identity with SEQ ID NO: 26, or

an amino acid sequence with one or (preferably 1, 2, 3, 4, 5, 6, 7, 8, 9 or 10) conservative amino acid mutations (preferably substitutions, insertions or deletions) compared to SEQ ID NO: 26;

(4) (i) a heavy chain variable region, which comprises or consists of the following sequence:

an amino acid sequence as shown in SEQ ID NO: 58, or

a sequence having at least 90%, preferably at least 91%, 92%, 93%, 94%, 95%, 96%, 97%, 98%, 99% or 100% sequence identity with SEQ ID NO: 58, or

an amino acid sequence with one or (preferably 1, 2, 3, 4, 5, 6, 7, 8, 9 or 10) conservative amino acid mutations (preferably substitutions, insertions or deletions) compared to SEQ ID NO: 58, and

(ii) a light chain variable region, which comprises or consists of the following sequence:

an amino acid sequence as shown in SEQ ID NO: 34, or

a sequence having at least 90%, preferably at least 91%, 92%, 93%, 94%, 95%, 96%, 97%, 98%, 99% or 100% sequence identity with SEQ ID NO: 34, or

an amino acid sequence with one or (preferably 1, 2, 3, 4, 5, 6, 7, 8, 9 or 10) conservative amino acid mutations (preferably substitutions, insertions or deletions) compared to SEQ ID NO: 34; or

(5) (i) a heavy chain variable region, which comprises or consists of the following sequence:

an amino acid sequence as shown in SEQ ID NO: 58, or

a sequence having at least 90%, preferably at least 91%, 92%, 93%, 94%, 95%, 96%, 97%, 98%, 99% or 100% sequence identity with SEQ ID NO: 58, or

an amino acid sequence with one or (preferably 1, 2, 3, 4, 5, 6, 7, 8, 9 or 10) conservative amino acid mutations (preferably substitutions, insertions or deletions) compared to SEQ ID NO: 58, and

(ii) a light chain variable region, which comprises or consists of the following sequence:

an amino acid sequence as shown in SEQ ID NO: 42, or

a sequence having at least 90%, preferably at least 91%, 92%, 93%, 94%, 95%, 96%, 97%, 98%, 99% or 100% sequence identity with SEQ ID NO: 42, or

an amino acid sequence with one or (preferably 1, 2, 3, 4, 42, 6, 7, 8, 9 or 10) conservative amino acid mutations (preferably substitutions, insertions or deletions) compared to SEQ ID NO: 42.

In a specific embodiment, the heavy chain variable region and the light chain variable region are respectively encoded by the following nucleotide sequences:

(1) (i) a nucleotide sequence as shown in SEQ ID NO: 22, or

a sequence having at least 90%, preferably at least 91%, 92%, 93%, 94%, 95%, 96%, 97%, 98%, 99% or 100% sequence identity with SEQ ID NO: 22, or

a nucleotide sequence with one or more (preferably 1, 2, 3, 4, 5, 6, 7, 8, 9 or 10) conservative mutations (preferably substitutions, insertions or deletions) compared to SEQ ID NO: 22, and

(ii) a nucleotide sequence as shown in SEQ ID NO: 23, or

a sequence having at least 90%, preferably at least 91%, 92%, 93%, 94%, 95%, 96%, 97%, 98%, 99% or 100% sequence identity with SEQ ID NO: 23, or

a nucleotide sequence with one or (preferably 1, 2, 3, 4, 5, 6, 7, 8, 9 or 10) conservative mutations (preferably substitutions, insertions or deletions) compared to SEQ ID NO: 23;

(2) (i) a nucleotide sequence as shown in SEQ ID NO: 51, or

a sequence having at least 90%, preferably at least 91%, 92%, 93%, 94%, 95%, 96%, 97%, 98%, 99% or 100% sequence identity with SEQ ID NO: 51, or

a nucleotide sequence with one or (preferably 1, 2, 3, 4, 5, 6, 7, 8, 9 or 10) conservative mutations (preferably substitutions, insertions or deletions) compared to SEQ ID NO: 51, and

(ii) a nucleotide sequence as shown in SEQ ID NO: 27, or

a sequence having at least 90%, preferably at least 91%, 92%, 93%, 94%, 95%, 96%, 97%, 98%, 99% or 100% sequence identity with SEQ ID NO: 27, or

a nucleotide sequence with one or (preferably 1, 2, 3, 4, 5, 6, 7, 8, 9 or 10) conservative mutations (preferably substitutions, insertions or deletions) compared to SEQ ID NO: 27;

(3) (i) a nucleotide sequence as shown in SEQ ID NO: 59, or

a sequence having at least 90%, preferably at least 91%, 92%, 93%, 94%, 95%, 96%, 97%, 98%, 99% or 100% sequence identity with SEQ ID NO: 59, or

a nucleotide sequence with one or (preferably 1, 2, 3, 4, 5, 6, 7, 8, 9 or 10) conservative mutations (preferably substitutions, insertions or deletions) compared to SEQ ID NO: 59, and

(ii) a nucleotide sequence as shown in SEQ ID NO: 27, or

a sequence having at least 90%, preferably at least 91%, 92%, 93%, 94%, 95%, 96%, 97%, 98%, 99% or 100% sequence identity with SEQ ID NO: 27, or

a nucleotide sequence with one or (preferably 1, 2, 3, 4, 5, 6, 7, 8, 9 or 10) conservative mutations (preferably substitutions, insertions or deletions) compared to SEQ ID NO: 27;

(4) (i) a nucleotide sequence as shown in SEQ ID NO: 59, or

a sequence having at least 90%, preferably at least 91%, 92%, 93%, 94%, 95%, 96%, 97%, 98%, 99% or 100% sequence identity with SEQ ID NO: 59, or

a nucleotide sequence with one or (preferably 1, 2, 3, 4, 5, 6, 7, 8, 9 or 10) conservative mutations (preferably substitutions, insertions or deletions) compared to SEQ ID NO: 59, and

(ii) a nucleotide sequence as shown in SEQ ID NO: 35, or

a sequence having at least 90%, preferably at least 91%, 92%, 93%, 94%, 95%, 96%, 97%, 98%, 99% or 100% sequence identity with SEQ ID NO: 35, or

a sequence with one or (preferably 1, 2, 3, 4, 5, 6, 7, 8, 9 or 10) conservative mutations (preferably substitutions, insertions or deletions) compared to SEQ ID NO: 35; or

(5) (i) a nucleotide sequence as shown in SEQ ID NO: 59, or

a sequence having at least 90%, preferably at least 91%, 92%, 93%, 94%, 95%, 96%, 97%, 98%, 99% or 100% sequence identity with SEQ ID NO: 59, or

a nucleotide sequence with one or (preferably 1, 2, 3, 4, 5, 6, 7, 8, 9 or 10) conservative mutations (preferably substitutions, insertions or deletions) compared to SEQ ID NO: 59, and

(ii) a nucleotide sequence as shown in SEQ ID NO: 43, or

a sequence having at least 90%, preferably at least 91%, 92%, 93%, 94%, 95%, 96%, 97%, 98%, 99% or 100% sequence identity with SEQ ID NO: 43, or

a nucleotide sequence with one or (preferably 1, 2, 3, 4, 5, 6, 7, 8, 9 or 10) conservative mutations (preferably substitutions, insertions or deletions) compared to SEQ ID NO: 43.

In a specific embodiment, the antibody further comprises framework regions FR-H1, FR-H2, FR-H3 and FR-H4 of the heavy chain variable region, and framework regions FR-L1, FR-L2, FR-L3 and FR-L4 of the light chain variable region, wherein

(1) FR-H1 comprises or consists of an amino acid sequence of SEQ ID NO: 12, or a sequence having at least 90%, preferably at least 91%, 92%, 93%, 94%, 95%, 96%, 97%, 98%, 99% or 100% sequence identity with SEQ ID NO: 12, or an amino acid sequence with one or (preferably 1, 2, 3, 4, 5, 6, 7, 8, 9 or 10) conservative amino acid mutations (preferably substitutions, insertions or deletions) compared to SEQ ID NO: 12;

FR-H2 comprises or consists of an amino acid sequence of SEQ ID NO: 13, or a sequence having at least 90%, preferably at least 91%, 92%, 93%, 94%, 95%, 96%, 97%, 98%, 99% or 100% sequence identity with SEQ ID NO: 13, or an amino acid sequence with one or (preferably 1, 2, 3, 4, 5, 6, 7, 8, 9 or 10) conservative amino acid mutations (preferably substitutions, insertions or deletions) compared to SEQ ID NO: 13;

FR-H3 comprises or consists of an amino acid sequence of SEQ ID NO: 14, or a sequence having at least 90%, preferably at least 91%, 92%, 93%, 94%, 95%, 96%, 97%, 98%, 99% or 100% sequence identity with SEQ ID NO: 14, or an amino acid sequence with one or (preferably 1, 2, 3, 4, 5, 6, 7, 8, 9 or 10) conservative amino acid mutations (preferably substitutions, insertions or deletions) compared to SEQ ID NO: 14;

FR-H4 comprises or consists of an amino acid sequence of SEQ ID NO: 15, or a sequence having at least 90%, preferably at least 91%, 92%, 93%, 94%, 95%, 96%, 97%, 98%, 99% or 100% sequence identity with SEQ ID NO: 15, or an amino acid sequence with one or (preferably 1, 2, 3, 4, 5, 6, 7, 8, 9 or 10) conservative amino acid mutations (preferably substitutions, insertions or deletions) compared to SEQ ID NO: 15; and

FR-L1 comprises or consists of an amino acid sequence of SEQ ID NO: 16, or a sequence having at least 90%, preferably at least 91%, 92%, 93%, 94%, 95%, 96%, 97%, 98%, 99% or 100% sequence identity with SEQ ID NO: 16, or an amino acid sequence with one or (preferably 1, 2, 3, 4, 5, 6, 7, 8, 9 or 10) conservative amino acid mutations (preferably substitutions, insertions or deletions) compared to SEQ ID NO: 16; FR-L2 comprises or consists of an amino acid sequence of SEQ ID NO: 17, or a sequence having at least 90%, preferably at least 91%, 92%, 93%, 94%, 95%, 96%, 97%, 98%, 99% or 100% sequence identity with SEQ ID NO: 17, or an amino acid sequence with one or (preferably 1, 2, 3, 4, 5, 6, 7, 8, 9 or 10) conservative amino acid mutations (preferably substitutions, insertions or deletions) compared to SEQ ID NO: 17; FR-L3 comprises or consists of an amino acid sequence of SEQ ID NO: 18, or a sequence having at least 90%, preferably at least 91%, 92%, 93%, 94%, 95%, 96%, 97%, 98%, 99% or 100% sequence identity with SEQ ID NO: 18, or an amino acid sequence with one or (preferably 1, 2, 3, 4, 5, 6, 7, 8, 9 or 10) conservative amino acid mutations (preferably substitutions, insertions or deletions) compared to SEQ ID NO: 18; FR-L4 comprises or consists of an amino acid sequence of SEQ ID NO: 19, or a sequence having at least 90%, preferably at least 91%, 92%, 93%, 94%, 95%, 96%, 97%, 98%, 99% or 100% sequence identity with SEQ ID NO: 19, or an amino acid sequence with one or (preferably 1, 2, 3, 4, 5, 6, 7, 8, 9 or 10) conservative amino acid mutations (preferably substitutions, insertions or deletions) compared to SEQ ID NO: 19;

(2) FR-H1 comprises or consists of an amino acid sequence of SEQ ID NO: 52, or a sequence having at least 90%, preferably at least 91%, 92%, 93%, 94%, 95%, 96%, 97%, 98%, 99% or 100% sequence identity with SEQ ID NO: 52, or an amino acid sequence with one or (preferably 1, 2, 3, 4, 5, 6, 7, 8, 9 or 10) conservative amino acid mutations (preferably substitutions, insertions or deletions) compared to SEQ ID NO: 52;

FR-H2 comprises or consists of an amino acid sequence of SEQ ID NO: 53, or a sequence having at least 90%, preferably at least 91%, 92%, 93%, 94%, 95%, 96%, 97%, 98%, 99% or 100% sequence identity with SEQ ID NO: 53, or an amino acid sequence with one or (preferably 1, 2, 3, 4, 5, 6, 7, 8, 9 or 10) conservative amino acid mutations (preferably substitutions, insertions or deletions) compared to SEQ ID NO: 53;

FR-H3 comprises or consists of an amino acid sequence of SEQ ID NO: 54, or a sequence having at least 90%, preferably at least 91%, 92%, 93%, 94%, 95%, 96%, 97%, 98%, 99% or 100% sequence identity with SEQ ID NO: 54, or an amino acid sequence with one or (preferably 1, 2, 3, 4, 5, 6, 7, 8, 9 or 10) conservative amino acid mutations (preferably substitutions, insertions or deletions) compared to SEQ ID NO: 54;

FR-H4 comprises or consists of an amino acid sequence of SEQ ID NO: 55, or a sequence having at least 90%, preferably at least 91%, 92%, 93%, 94%, 95%, 96%, 97%, 98%, 99% or 100% sequence identity with SEQ ID NO: 55, or an amino acid sequence with one or (preferably 1, 2, 3, 4, 5, 6, 7, 8, 9 or 10) conservative amino acid mutations (preferably substitutions, insertions or deletions) compared to SEQ ID NO: 55; and

FR-L1 comprises or consists of an amino acid sequence of SEQ ID NO: 28, or a sequence having at least 90%, preferably at least 91%, 92%, 93%, 94%, 95%, 96%, 97%, 98%, 99% or 100% sequence identity with SEQ ID NO: 28, or an amino acid sequence with one or (preferably 1, 2, 3, 4, 5, 6, 7, 8, 9 or 10) conservative amino acid mutations (preferably substitutions, insertions or deletions) compared to SEQ ID NO: 28;

FR-L2 comprises or consists of an amino acid sequence of SEQ ID NO: 29, or a sequence having at least 90%, preferably at least 91%, 92%, 93%, 94%, 95%, 96%, 97%, 98%, 99% or 100% sequence identity with SEQ ID NO: 29, or an amino acid sequence with one or (preferably 1, 2, 3, 4, 5, 6, 7, 8, 9 or 10) conservative amino acid mutations (preferably substitutions, insertions or deletions) compared to SEQ ID NO: 29;

FR-L3 comprises or consists of an amino acid sequence of SEQ ID NO: 30, or a sequence having at least 90%, preferably at least 91%, 92%, 93%, 94%, 95%, 96%, 97%, 98%, 99% or 100% sequence identity with SEQ ID NO: 30, or an amino acid sequence with one or (preferably 1, 2, 3, 4, 5, 6, 7, 8, 9 or 10) conservative amino acid mutations (preferably substitutions, insertions or deletions) compared to SEQ ID NO: 30;

FR-L4 comprises or consists of an amino acid sequence of SEQ ID NO: 31, or a sequence having at least 90%, preferably at least 91%, 92%, 93%, 94%, 95%, 96%, 97%, 98%, 99% or 100% sequence identity with SEQ ID NO: 31, or an amino acid sequence with one or (preferably 1, 2, 3, 4, 5, 6, 7, 8, 9 or 10) conservative amino acid mutations (preferably substitutions, insertions or deletions) compared to SEQ ID NO: 31;

(3) FR-H1 comprises or consists of an amino acid sequence of SEQ ID NO: 60, or a sequence having at least 90%, preferably at least 91%, 92%, 93%, 94%, 95%, 96%, 97%, 98%, 99% or 100% sequence identity with SEQ ID NO: 60, or an amino acid sequence with one or (preferably 1, 2, 3, 4, 5, 6, 7, 8, 9 or 10) conservative amino acid mutations (preferably substitutions, insertions or deletions) compared to SEQ ID NO: 60;

FR-H2 comprises or consists of an amino acid sequence of SEQ ID NO: 61, or a sequence having at least 90%, preferably at least 91%, 92%, 93%, 94%, 95%, 96%, 97%, 98%, 99% or 100% sequence identity with SEQ ID NO: 61, or an amino acid sequence with one or (preferably 1, 2, 3, 4, 5, 6, 7, 8, 9 or 10) conservative amino acid mutations (preferably substitutions, insertions or deletions) compared to SEQ ID NO: 61;

FR-H3 comprises or consists of an amino acid sequence of SEQ ID NO: 62, or a sequence having at least 90%, preferably at least 91%, 92%, 93%, 94%, 95%, 96%, 97%, 98%, 99% or 100% sequence identity with SEQ ID NO: 62, or an amino acid sequence with one or (preferably 1, 2, 3, 4, 5, 6, 7, 8, 9 or 10) conservative amino acid mutations (preferably substitutions, insertions or deletions) compared to SEQ ID NO: 62;

FR-H4 comprises or consists of an amino acid sequence of SEQ ID NO: 63, or a sequence having at least 90%, preferably at least 91%, 92%, 93%, 94%, 95%, 96%, 97%, 98%, 99% or 100% sequence identity with SEQ ID NO: 63, or an amino acid sequence with one or (preferably 1, 2, 3, 4, 5, 6, 7, 8, 9 or 10) conservative amino acid mutations (preferably substitutions, insertions or deletions) compared to SEQ ID NO: 63; and

FR-L1 comprises or consists of an amino acid sequence of SEQ ID NO: 28, or a sequence having at least 90%, preferably at least 91%, 92%, 93%, 94%, 95%, 96%, 97%, 98%, 99% or 100% sequence identity with SEQ ID NO: 28, or an amino acid sequence with one or (preferably 1, 2, 3, 4, 5, 6, 7, 8, 9 or 10) conservative amino acid mutations (preferably substitutions, insertions or deletions) compared to SEQ ID NO: 28;

FR-L2 comprises or consists of an amino acid sequence of SEQ ID NO: 29, or a sequence having at least 90%, preferably at least 91%, 92%, 93%, 94%, 95%, 96%, 97%, 98%, 99% or 100% sequence identity with SEQ ID NO: 29, or an amino acid sequence with one or (preferably 1, 2, 3, 4, 5, 6, 7, 8, 9 or 10) conservative amino acid mutations (preferably substitutions, insertions or deletions) compared to SEQ ID NO: 29;

FR-L3 comprises or consists of an amino acid sequence of SEQ ID NO: 30, or a sequence having at least 90%, preferably at least 91%, 92%, 93%, 94%, 95%, 96%, 97%, 98%, 99% or 100% sequence identity with SEQ ID NO: 30, or an amino acid sequence with one or (preferably 1, 2, 3, 4, 5, 6, 7, 8, 9 or 10) conservative amino acid mutations (preferably substitutions, insertions or deletions) compared to SEQ ID NO: 30;

FR-L4 comprises or consists of an amino acid sequence of SEQ ID NO: 31, or a sequence having at least 90%, preferably at least 91%, 92%, 93%, 94%, 95%, 96%, 97%, 98%, 99% or 100% sequence identity with SEQ ID NO: 31, or an amino acid sequence with one or (preferably 1, 2, 3, 4, 5, 6, 7, 8, 9 or 10) conservative amino acid mutations (preferably substitutions, insertions or deletions) compared to SEQ ID NO: 31;

(4) FR-H1 comprises or consists of an amino acid sequence of SEQ ID NO: 60, or a sequence having at least 90%, preferably at least 91%, 92%, 93%, 94%, 95%, 96%, 97%, 98%, 99% or 100% sequence identity with SEQ ID NO: 60, or an amino acid sequence with one or (preferably 1, 2, 3, 4, 5, 6, 7, 8, 9 or 10) conservative amino acid mutations (preferably substitutions, insertions or deletions) compared to SEQ ID NO: 60;

FR-H2 comprises or consists of an amino acid sequence of SEQ ID NO: 61, or a sequence having at least 90%, preferably at least 91%, 92%, 93%, 94%, 95%, 96%, 97%, 98%, 99% or 100% sequence identity with SEQ ID NO: 61, or an amino acid sequence with one or (preferably 1, 2, 3, 4, 5, 6, 7, 8, 9 or 10) conservative amino acid mutations (preferably substitutions, insertions or deletions) compared to SEQ ID NO: 61;

FR-H3 comprises or consists of an amino acid sequence of SEQ ID NO: 62, or a sequence having at least 90%, preferably at least 91%, 92%, 93%, 94%, 95%, 96%, 97%, 98%, 99% or 100% sequence identity with SEQ ID NO: 62, or an amino acid sequence with one or (preferably 1, 2, 3, 4, 5, 6, 7, 8, 9 or 10) conservative amino acid mutations (preferably substitutions, insertions or deletions) compared to SEQ ID NO: 62;

FR-H4 comprises or consists of an amino acid sequence of SEQ ID NO: 63, or a sequence having at least 90%, preferably at least 91%, 92%, 93%, 94%, 95%, 96%, 97%, 98%, 99% or 100% sequence identity with SEQ ID NO: 63, or an amino acid sequence with one or (preferably 1, 2, 3, 4, 5, 6, 7, 8, 9 or 10) conservative amino acid mutations (preferably substitutions, insertions or deletions) compared to SEQ ID NO: 63; and

FR-L1 comprises or consists of an amino acid sequence of SEQ ID NO: 36, or a sequence having at least 90%, preferably at least 91%, 92%, 93%, 94%, 95%, 96%, 97%, 98%, 99% or 100% sequence identity with SEQ ID NO: 36, or an amino acid sequence with one or (preferably 1, 2, 3, 4, 5, 6, 7, 8, 9 or 10) conservative amino acid mutations (preferably substitutions, insertions or deletions) compared to SEQ ID NO: 36;

FR-L2 comprises or consists of an amino acid sequence of SEQ ID NO: 37, or a sequence having at least 90%, preferably at least 91%, 92%, 93%, 94%, 95%, 96%, 97%, 98%, 99% or 100% sequence identity with SEQ ID NO: 37, or an amino acid sequence with one or (preferably 1, 2, 3, 4, 5, 6, 7, 8, 9 or 10) conservative amino acid mutations (preferably substitutions, insertions or deletions) compared to SEQ ID NO: 37;

FR-L3 comprises or consists of an amino acid sequence of SEQ ID NO: 38, or a sequence having at least 90%, preferably at least 91%, 92%, 93%, 94%, 95%, 96%, 97%, 98%, 99% or 100% sequence identity with SEQ ID NO: 38, or an amino acid sequence with one or (preferably 1, 2, 3, 4, 5, 6, 7, 8, 9 or 10) conservative amino acid mutations (preferably substitutions, insertions or deletions) compared to SEQ ID NO: 38;

FR-L4 comprises or consists of an amino acid sequence of SEQ ID NO: 39, or a sequence having at least 90%, preferably at least 91%, 92%, 93%, 94%, 95%, 96%, 97%, 98%, 99% or 100% sequence identity with SEQ ID NO: 39, or an amino acid sequence with one or (preferably 1, 2, 3, 4, 5, 6, 7, 8, 9 or 10) conservative amino acid mutations (preferably substitutions, insertions or deletions) compared to SEQ ID NO: 39; or

(5) FR-H1 comprises or consists of an amino acid sequence of SEQ ID NO: 60, or a sequence having at least 90%, preferably at least 91%, 92%, 93%, 94%, 95%, 96%, 97%, 98%, 99% or 100% sequence identity with SEQ ID NO: 60, or an amino acid sequence with one or (preferably 1, 2, 3, 4, 5, 6, 7, 8, 9 or 10) conservative amino acid mutations (preferably substitutions, insertions or deletions) compared to SEQ ID NO: 60;

FR-H2 comprises or consists of an amino acid sequence of SEQ ID NO: 61, or a sequence having at least 90%, preferably at least 91%, 92%, 93%, 94%, 95%, 96%, 97%, 98%, 99% or 100% sequence identity with SEQ ID NO: 61, or an amino acid sequence with one or (preferably 1, 2, 3, 4, 5, 6, 7, 8, 9 or 10) conservative amino acid mutations (preferably substitutions, insertions or deletions) compared to SEQ ID NO: 61;

FR-H3 comprises or consists of an amino acid sequence of SEQ ID NO: 62, or a sequence having at least 90%, preferably at least 91%, 92%, 93%, 94%, 95%, 96%, 97%, 98%, 99% or 100% sequence identity with SEQ ID NO: 62, or an amino acid sequence with one or (preferably 1, 2, 3, 4, 5, 6, 7, 8, 9 or 10) conservative amino acid mutations (preferably substitutions, insertions or deletions) compared to SEQ ID NO: 62;

FR-H4 comprises or consists of an amino acid sequence of SEQ ID NO: 63, or a sequence having at least 90%, preferably at least 91%, 92%, 93%, 94%, 95%, 96%, 97%, 98%, 99% or 100% sequence identity with SEQ ID NO: 63, or an amino acid sequence with one or (preferably 1, 2, 3, 4, 5, 6, 7, 8, 9 or 10) conservative amino acid mutations (preferably substitutions, insertions or deletions) compared to SEQ ID NO: 63; and

FR-L1 comprises or consists of an amino acid sequence of SEQ ID NO: 44, or a sequence having at least 90%, preferably at least 91%, 92%, 93%, 94%, 95%, 96%, 97%, 98%, 99% or 100% sequence identity with SEQ ID NO: 44, or an amino acid sequence with one or (preferably 1, 2, 3, 4, 5, 6, 7, 8, 9 or 10) conservative amino acid mutations (preferably substitutions, insertions or deletions) compared to SEQ ID NO: 44;

FR-L2 comprises or consists of an amino acid sequence of SEQ ID NO: 45, or a sequence having at least 90%, preferably at least 91%, 92%, 93%, 94%, 95%, 96%, 97%, 98%, 99% or 100% sequence identity with SEQ ID NO: 45, or an amino acid sequence with one or (preferably 1, 2, 3, 4, 5, 6, 7, 8, 9 or 10) conservative amino acid mutations (preferably substitutions, insertions or deletions) compared to SEQ ID NO: 45;

FR-L3 comprises or consists of an amino acid sequence of SEQ ID NO: 46, or a sequence having at least 90%, preferably at least 91%, 92%, 93%, 94%, 95%, 96%, 97%, 98%, 99% or 100% sequence identity with SEQ ID NO: 46, or an amino acid sequence with one or (preferably 1, 2, 3, 4, 5, 6, 7, 8, 9 or 10) conservative amino acid mutations (preferably substitutions, insertions or deletions) compared to SEQ ID NO: 46;

FR-L4 comprises or consists of an amino acid sequence of SEQ ID NO: 47, or a sequence having at least 90%, preferably at least 91%, 92%, 93%, 94%, 95%, 96%, 97%, 98%, 99% or 100% sequence identity with SEQ ID NO: 47, or an amino acid sequence with one or (preferably 1, 2, 3, 4, 5, 6, 7, 8, 9 or 10) conservative amino acid mutations (preferably substitutions, insertions or deletions) compared to SEQ ID NO: 47.

In a specific embodiment, the antibody comprises or consists of an amino acid sequence with the following heavy chain and light chain combinations:

(1) an amino acid sequence of SEQ ID NO: 2 and an amino acid sequence of SEQ ID NO: 3;

(2) an amino acid sequence of SEQ ID NO: 48 and an amino acid sequence of SEQ ID NO: 24;

(3) an amino acid sequence of SEQ ID NO: 56 and an amino acid sequence of SEQ ID NO: 24;

(4) an amino acid sequence of SEQ ID NO: 56 and an amino acid sequence of SEQ ID NO: 32; or

(5) an amino acid sequence of SEQ ID NO: 56 and an amino acid sequence of SEQ ID NO: 40.

In a specific embodiment, the heavy chain and the light chain are respectively encoded by the following nucleotide sequences:

(1) a nucleotide sequence of SEQ ID NO: 20 and a nucleotide sequence of SEQ ID NO: 21;

(2) a nucleotide sequence of SEQ ID NO: 49 and a nucleotide sequence of SEQ ID NO: 25;

(3) a nucleotide sequence of SEQ ID NO: 57 and a nucleotide sequence of SEQ ID NO: 25;

(4) a nucleotide sequence of SEQ ID NO: 57 and a nucleotide sequence of SEQ ID NO: 33; or

(5) a nucleotide sequence of SEQ ID NO: 57 and a nucleotide sequence of SEQ ID NO: 41.

In specific embodiments, the antibody is a humanized antibody, a chimeric antibody, or a multispecific antibody (e.g., a bispecific antibody).

In a specific embodiment, the constant region of the antibody is humanized, preferably derived from human IgG, more preferably derived from human IgG1 or IgG4.

In a specific embodiment, the heavy chain constant region of the antibody adopts an Ig gamma-1 or Ig gamma-4 chain C region, preferably an Ig gamma-1 chain C region; the light chain constant region adopts an Ig kappa chain C region, more preferably an Ig kappa chain C region of the GenBank accession number ACCESSION: P01834.

In a specific embodiment, the antigen-binding fragment is selected from Fab, Fab′, F(ab′)2, Fd, Fv, dAb, Fab/c, complementarity determining region (CDR) fragment, single-chain antibody (for example, scFv), bivalent antibody or domain antibody.

In another aspect of the present invention, it relates to an isolated polypeptide, which is selected from the group consisting of:

(1) an isolated polypeptide comprising the sequence shown in SEQ ID NOs: 6, 7 and 8, wherein the polypeptide is a part of an antibody against human IL-6 and specifically binds to human IL-6, and the antibody also contains the sequences shown in SEQ ID NOs: 9, 10 and 11;

(2) an isolated polypeptide comprising the sequence shown in SEQ ID NOs: 9, 10 and 11, wherein the polypeptide is a part of an antibody against human IL-6 and specifically binds to human IL-6, and the antibody also contains the sequences shown in SEQ ID NOs: 6, 7 and 8;

(3) an isolated polypeptide, which comprises a sequence selected from SEQ ID NO: 4 or 50, or a sequence having at least 90%, preferably at least 91%, 92%, 93%, 94%, 95%, 96%, 97%, 98%, 99% or 100% sequence identity with SEQ ID NO: 4 or 50, or an amino acid sequence with one or (preferably 1, 2, 3, 4, 5, 6, 7, 8, 9 or 10) conservative amino acid mutations (preferably substitutions, insertions or deletions) compared to SEQ ID NO: 4 or 50, wherein the polypeptide, as a part of an antibody against human IL-6, specifically binds to human IL-6, and the antibody also includes a sequence selected from SEQ ID NO: 5 or 26, or a sequence having at least 90%, preferably at least 91%, 92%, 93%, 94%, 95%, 96%, 97%, 98%, 99% or 100% sequence identity with SEQ ID NO: 5 or 26, or an amino acid sequence with one or (preferably 1, 2, 3, 4, 5, 6, 7, 8, 9 or 10) conservative amino acid mutations (preferably substitutions, insertions or deletions) compared to SEQ ID NO: 5 or 26;

(4) an isolated polypeptide, which comprises a sequence shown in SEQ ID NO: 58, or a sequence having at least 90%, preferably at least 91%, 92%, 93%, 94%, 95%, 96%, 97%, 98%, 99% or 100% sequence identity with SEQ ID NO: 58, or an amino acid sequence with one or (preferably 1, 2, 3, 4, 5, 6, 7, 8, 9 or 10) conservative amino acid mutations (preferably substitutions, insertions or deletions) compared to SEQ ID NO: 58, wherein the polypeptide, as a part of an antibody against human IL-6, specifically binds to human IL-6, and the antibody also includes a sequence selected from SEQ ID NO: 26, 34 or 42, or a sequence having at least 90%, preferably at least 91%, 92%, 93%, 94%, 95%, 96%, 97%, 98%, 99% or 100% sequence identity with SEQ ID NO: 26, 34 or 42, or an amino acid sequence with one or (preferably 1, 2, 3, 4, 5, 6, 7, 8, 9 or 10) conservative amino acid mutations (preferably substitutions, insertions or deletions) compared to SEQ ID NO: 26, 34 or 42;

(5) an isolated polypeptide, which comprises a sequence shown in SEQ ID NO: 5 or 26, or a sequence having at least 90%, preferably at least 91%, 92%, 93%, 94%, 95%, 96%, 97%, 98%, 99% or 100% sequence identity with SEQ ID NO: 5 or 26, or an amino acid sequence with one or (preferably 1, 2, 3, 4, 5, 6, 7, 8, 9 or 10) conservative amino acid mutations (preferably substitutions, insertions or deletions) compared to SEQ ID NO: 5 or 26, wherein the polypeptide, as a part of an antibody against human IL-6, specifically binds to human IL-6, and the antibody also includes a sequence selected from SEQ ID NO: 4 or 50, or a sequence having at least 90%, preferably at least 91%, 92%, 93%, 94%, 95%, 96%, 97%, 98%, 99% or 100% sequence identity with SEQ ID NO: 4 or 50, or an amino acid sequence with one or (preferably 1, 2, 3, 4, 5, 6, 7, 8, 9 or 10) conservative amino acid mutations (preferably substitutions, insertions or deletions) compared to SEQ ID NO: 4 or 50; or

(6) an isolated polypeptide, which comprises a sequence shown in SEQ ID NO: 26, 34 or 42, or a sequence having at least 90%, preferably at least 91%, 92%, 93%, 94%, 95%, 96%, 97%, 98%, 99% or 100% sequence identity with SEQ ID NO: 26, 34 or 42, or an amino acid sequence with one or (preferably 1, 2, 3, 4, 5, 6, 7, 8, 9 or 10) conservative amino acid mutations (preferably substitutions, insertions or deletions) compared to SEQ ID NO: 26, 34 or 42, wherein the polypeptide, as a part of an antibody against human IL-6, specifically binds to human IL-6, and the antibody also includes a sequence selected from SEQ ID NO: 58, or a sequence having at least 90%, preferably at least 91%, 92%, 93%, 94%, 95%, 96%, 97%, 98%, 99% or 100% sequence identity with SEQ ID NO: 58, or an amino acid sequence with one or (preferably 1, 2, 3, 4, 5, 6, 7, 8, 9 or 10) conservative amino acid mutations (preferably substitutions, insertions or deletions) compared to SEQ ID NO: 58.

In another aspect, the present invention relates to an isolated polynucleotide, which encodes the isolated polypeptide of the present invention.

In another aspect, the present invention relates to a vector, which comprises the isolated polynucleotide of the present invention.

In another aspect, the present invention relates to a host cell, which comprises the isolated polynucleotide of the present invention or the vector of the present invention.

In another aspect, the present invention relates to a method for preparing the antibody or antigen-binding fragment thereof of the present invention, including culturing the host cell of the present invention.

In another aspect, the present invention relates to an antibody conjugate, which comprises the antibody or antigen-binding fragment thereof of the present invention and a coupling moiety coupled thereto, preferably, the coupling moiety is selected from purification tags (such as His tag), cytotoxic agents, detectable labels, radioisotopes, luminescent substances, colored substances, enzymes or polyethylene glycol.

In another aspect, the present invention relates to a multispecific antibody, preferably a bispecific antibody, which includes the antibody or antigen-binding fragment thereof of the present invention, as well as an antibody or antigen-binding fragment against other antigens and/or other epitopes.

In another aspect, the present invention relates to a fusion protein, which comprises the antibody or antigen-binding fragment thereof of the present invention.

In another aspect, the present invention relates to a pharmaceutical composition, which comprises the antibody or antigen-binding fragment thereof, the antigen conjugate, the multispecific antibody or the fusion protein of the present invention, and optionally a pharmaceutically acceptable carrier and/or excipient.

In a specific embodiment, the pharmaceutical composition is a dosage form suitable for oral administration to the gastrointestinal (GI) tract, preferably at least one dosage form administrated in a mode selected from parenteral administration, subcutaneous administration, intramuscular administration, intravenous administration, intraarticular administration, intrabronchial administration, intraabdominal administration, intravesicular administration, intrachondral administration, intracavitary administration, intra-bodycavitary administration, intracerebellar administration, intracerebroventricular administration, intracolonic administration, intra-neck administration, intragastric administration, intrahepatic administration, intramyocardial administration, intraosseous administration, intrapelvic administration, intrapericardial administration, intraperitoneum administration, intrapleural administration, intraprostatic administration, intrapulmonary administration, intrarectal administration, intrarenal administration, intraretinal administration, intraspinal administration, intrasynovial administration, intrathoracic administration, intrauterine administration, intravesical administration, bolus administration, intravaginal administration, rectal administration, buccal administration, sublingual administration, intranasal administration, or transdermal administration.

In another aspect, the present invention relates to a kit comprising the antibody or antigen-binding fragment thereof, the antigen conjugate, the multispecific antibody or the fusion protein of the present invention, preferably the kit also includes a second antibody, which specifically targets the antibody or antigen-binding fragment thereof, the antigen conjugate, the multispecific antibody or the fusion protein; optionally, the second antibody also includes a detectable label, such as a radioisotope, a luminescent substance, a colored substance, and an enzyme.

In another aspect, the present invention relates to use of the antibody or antigen-binding fragment thereof, the antigen conjugate, the multispecific antibody or the fusion protein of the present invention in the preparation of a kit, wherein the kit is used to detect the presence or level of human IL-6 in a sample.

In another aspect, the present invention relates to the use of the antibody or antigen-binding fragment thereof, the antigen conjugate, the multispecific antibody or the fusion protein of the present invention in the preparation of the following medicaments:

a medicament that blocks the binding of human IL-6 to human IL-6R,

a medicament that blocks the activity of human IL-6 or down-regulates the level of human IL-6,

a medicament that inhibits the signal transduction of gp130 and reduces the phosphorylation level of its downstream signal protein p-Stat3 (Tyr705), or

a medicament that blocks the cytobiological response mediated by the binding of human IL-6 and IL-6R.

In another aspect, the present invention relates to the use of the antibody or antigen-binding fragment thereof, the antigen conjugate, the multispecific antibody or the fusion protein of the present invention in the preparation of a medicament for prevention and/or treatment and/or adjuvant treatment and/or diagnosis of IL-6-related diseases.

In another aspect, the present invention relates to the antibody or antigen-binding fragment thereof, the antigen conjugate, the multispecific antibody or the fusion protein of the present invention, which is used for prevention and/or treatment and/or adjuvant treatment and/or diagnosis of IL-6-related diseases.

In another aspect, the present invention relates to an in vivo or in vitro method, comprising a step of administrating a cell containing the antibody or antigen-binding fragment thereof, the antigen conjugate, the multispecific antibody or the fusion protein of the present invention, or a step of administering an effective amount of the antibody or antigen-binding fragment thereof, the antigen conjugate, the multispecific antibody or the fusion protein to a subject in need thereof, wherein the method is used to

block the binding of human IL-6 to human IL-6R,

block the activity of human IL-6 or down-regulate the level of human IL-6,

inhibit the signal transduction of gp130 and reduce the phosphorylation level of its downstream signal protein p-Stat3 (Tyr705), or

block the cytobiological response mediated by the binding of human IL-6 and IL-6R.

In another aspect, the present invention relates to a method for prevention and/or treatment and/or adjuvant treatment and/or diagnosis of IL-6 related diseases, comprising administering the antibody or antigen-binding fragment, the antigen conjugate, the multispecific antibody or the fusion protein of the present invention to a subject in need thereof.

In some embodiments, the IL-6-related diseases include, but are not limited to, at least one of obesity, immune-related diseases, cardiovascular diseases, infectious diseases, malignancies, neurological diseases, wound, trauma or tissue damage or related chronic conditions.

The IL-6-related immune-related diseases include but are not limited to at least one of the following:

(1) Respiratory Diseases

obstructive airway disease; asthma; bronchitis; acute rhinitis, allergic rhinitis, atrophic rhinitis and chronic rhinitis; membranous rhinitis; seasonal rhinitis; sarcoidosis, farmer's lung and related diseases, adult respiratory distress syndrome, allergic pneumonia, fibrotic lung and idiopathic interstitial pneumonia; neonatal chronic lung disease;

(2) Bone and Joint Diseases

rheumatoid arthritis, childhood rheumatoid arthritis, systemic juvenile rheumatoid arthritis, juvenile chronic arthritis, seronegative spondyloarthritis (including psoriatic arthritis, ankylosing spondylitis and Wright's disease), Behcet's disease, Sjogren's syndrome, systemic sclerosis, osteoarthritis, gout, osteolysis;

(3) Skin Diseases

psoriasis, allergic contact dermatitis, contact dermatitis, atopic dermatitis, other eczema skin diseases, seborrheic dermatitis, lichen planus, scleroderma, pemphigus, bullous pemphigoid, epidermolysis bullosa, urticaria, rubella, xerodermas (angiodermas), vasculitis, erythema, skin eosinophilia, uveitis, alopecia areata, allergic conjunctivitis and vernal vemal conjunctivitis;

(4) Gastrointestinal Tract Diseases

Stomach ulcer, inflammatory bowel disease, ulcerative colitis, abdominal disease, proctitis, eosinophilic gastroenteritis, mastocytosis, Crohn's disease, ulcerative colitis, antiphospholipid syndrome, food-related allergies leading to effects far away from internal organs such as migraine, rhinitis and eczema;

(5) graft Rejection

graft, Graft versus host disease, allograft rejection of any organ or tissue (kidney, heart, liver, pancreas, lung, bone marrow, skin, cartilage, bone, small intestine, fetal thymus, parathyroid, cornea), xenograft rejection of any organ or tissue;

(6) Other Tissues and Systemic Diseases

cachexia, systemic lupus erythematosus, skin lupus erythematosus, lupus nephritis, antiphospholipid syndrome, iridocyclitis/uveitis/optic neuritis, systemic vasculitis/Wegener's granulomatosis, Sarcoidosis, orchitis/vasectomy reversal process, allergic/atopic disease, allergic contact dermatitis, systemic inflammatory response syndrome, sepsis syndrome, gram-positive sepsis, gram-negative sepsis, culture Negative sepsis, fungal sepsis, neutropenia fever, urinary sepsis, meningococcal bacteremia, trauma/bleeding, burns, ionizing radiation exposure, acute pancreatitis, alcohol-induced hepatitis, chronic inflammatory pathology, aseptic relaxation of surgical implants, Sarcoidosis, sickle cell anemia, diabetes, nephropathy, atopic disease, hypersensitivity, hay fever, endometriosis, systemic anaphylaxis, pernicious anemia, hemolytic disease, thrombocytopenia, anti-receptor hypersensitivity, Graves' disease, Raynaud's disease, type B insulin resistant diabetes, myasthenia gravis, antibody-mediated cytotoxicity, type III hypersensitivity, POEMS syndrome (polyneuropathy, megaorganism, endocrine disease, monoclonal gammopathy and skin change syndrome), polyneuropathy, megaorganism, endocrine disease, monoclonal gammopathy, skin change syndrome, antiphospholipid symptoms, mixed connective tissue disease, primary Addison's disease, chronic active hepatitis, primary biliary cirrhosis, leukoplakia, vasculitis, post-MI cardiotomy syndrome, type IV allergies, granulomas caused by intracellular organisms, drug allergy, metabolic diseases/spontaneous disease, Wilson's disease, hemochromatosis, α-1-antitrypsin deficiency, diabetic retinopathy, Hashimoto's thyroiditis, hypothalamus-Pituitary-adrenal axis assessment, primary biliary cirrhosis, thyroiditis, encephalomyelitis, cystic fibrosis, familial hematophagocytic lymphohistiocytosis, skin medication disorders, nephrotic syndrome, nephritis, glomerulonephritis, acute renal failure, hemodialysis, uremia, poisoning, preeclampsia, okt3 therapy, anti-CD3 therapy, alopecia, cytokine therapy, chemotherapy, radiotherapy (such as including but not limited to fatigue, anemia, cachexia, etc.), chronic salicylate poisoning, etc.

See, for example, Merck Manual, Edition 12-17, Merck & Company, Rahway, N.J. (1972, 1977, 1982, 1987, 1992, 1999), Pharmacotherapy Handbook, Wells et al., 2nd Edition, Appleton and Lange, Stamford, Conn. (1998, 2000), all of which are fully incorporated by reference.

The cardiovascular diseases include but are not limited to at least one of the following diseases: cardiacstun syndrome, myocardial infarction, congestive heart failure, stroke, ischemic attack, hemorrhage, acute coronary syndrome, arteriosclerosis, atherosclerosis, restenosis, diabetes, diabetic macular edema, diabetic aterosclerotic disease, hypertension, arterial hypertension, renovascular hypertension, syncope, shock, cardiovascular system syphilis, heart failure, pulmonary (primary) heart disease, primary pulmonary hypertension, arrhythmia, atrial ectopic beats, atrial flutter, atrial fibrillation (persistent or sudden), post-perfusion syndrome, cardiopulmonary bypass inflammation, disturbed or multi-source atrial tachycardia, regular narrow QRS tachycardia, special arrhythmia, ventricular fibrillation, His bundle arrythmias, atrioventricular block, bundle branch block, myocardial ischemic disease, coronary artery disease, angina pectoris, myocardial infarction, cardiomyopathy, dilated congestive cardiomyopathy, restrictive cardiomyopathy, heart valve disease, endocarditis, pericardial disease, heart tumor, aorta and peripheral aneurysm, aortic wall dissection, aortic inflammation, abdominal aorta and its branch occlusion, peripheral vascular disease, occlusive artery disease, peripheral atherlosclerotic disease, thromboangiitis obliterans, functional peripheral artery disease, Raynaud's phenomenon and disease, hand and foot cyanosis, erythematous limb pain, venous disease, thrombophlebitis, varicose vein, arteriovenous fistula, lymphedema, lipoedema, unstable angina, reperfusion injury, post pump syndrome, ischemia-reperfusion injury, etc. Such methods may optionally include administering an effective amount of the composition or pharmaceutical composition comprising at least one anti-IL-6 antibody to the cells, tissues, organs, animals or patients in need of such modulation, treatment or therapy.

The IL-6-related infectious diseases include but are not limited to at least one of the following diseases: acute or chronic bacterial infection, acute or chronic parasitic or infectious process, including bacterial, viral and fungal infections, HTV infection/HIV neuropathy, meningitis, Hepatitis (such as type A, type B or type C, etc.), septic arthritis, peritonitis, pneumonia, epiglottitis, E. coli 0157:h7, hemolytic uremic syndrome/thrombolytic thrombocytopenic purpura, malaria, dengue Leather hemorrhagic fever, leishmaniasis, leprosy, toxic shock syndrome, streptococcal myositis, gas gangrene, Mycobacterium tuberculosis, Mycobacterium avium intracellular parasites, Pneumocystis carinii pneumonia, pelvic inflammatory disease, orchitis/epididymitis, Legionella, Lyme disease, influenza A, Epstein-Barr virus, virus-related hemophagocytic syndrome, viral encephalitis/sterile meningitis, enterovirus 71 hand, foot and mouth disease, etc.

The IL-6-related malignancies include but are not limited to at least one of the following diseases: leukemia, acute leukemia, acute lymphoblastic leukemia (ALL), acute lymphocytic leukemia, B-cell, T-cell or FAB ALL, Acute Myelogenous Leukemia (AML), Acute Myeloid Leukemia, Chronic Myeloid Leukemia (CML), Chronic Lymphocytic Leukemia (CLL), Hairy Cell Leukemia, Myelodysplastic Syndrome (MDS), Lymphoma, Hodgkin Disease, malignant lymphoma, non-Hodgkin's lymphoma, Burkitt's lymphoma, multiple myeloma, Kaposi's sarcoma, colorectal cancer, pancreatic cancer, nasopharyngeal carcinoma, malignant histiocytosis, paraneoplastic syndrome/malignant hypercalcemia (idiopathic) syndrome, solid tumors, bladder cancer, breast cancer, colorectal cancer, endometrial cancer, head cancer, neck cancer, hereditary non-polyposis cancer, Hodgkin's Lymphoma, liver cancer, lung cancer, non-small cell lung cancer, ovarian cancer, pancreatic cancer, prostate cancer, renal cell carcinoma, testicular cancer, adenocarcinoma, sarcoma, malignant melanoma, hemangioma, tumor metastatic disease, cancer-related bone resorption, cancer-related bone pain, etc.; inhibition of cancer metastasis; improvement of cancer cachexia.

The IL-6-related neurological diseases include but are not limited to at least one of the following diseases: neurodegenerative diseases, multiple sclerosis, migraine, AIDS dementia syndrome, demyelinating diseases, such as multiple sclerosis and acute transverse myelitis; extrapyramidal and cerebellar disorders, such as corticospinal system damage; basal ganglia disorders; hyperkinetic dyskinesias, such as Huntington's chorea and senile chorea; drug-induced dyskinesia, such as diseases induced by drugs that block CNS dopamine receptors; hypokinetic dyskinesias, such as Parkinson's disease; Progressive supranucleo Palsy; cerebellar structural damage; spinocerebellar degeneration, such as spinal ataxia, Freeh Dreich's ataxia, cerebellar cortex degeneration, multiple system degenerations (Mencel, Dejerine-Thomas, Shi-Drager and Machado-Joseph); systemic disorders (Raphthum's disease, abetalipoprotemia, ataxia, telangiectasia and mitochondrial multisystem disorders); demyelinating core disorders, such as multiple sclerosis, acute transverse myelitis; and motor unit disorders such as neuromuscular atrophy (anterior horn cell degeneration, such as amyotrophic (spinal cord) lateral sclerosis, infantile spinal muscular atrophy, and juvenile spinal muscular atrophy); Alzheimer's disease; Down syndrome in middle-aged people; diffuse Lewy body disease; Lewy body type senile dementia; Wernicke-Korsakov syndrome; chronic alcoholism; Kreuzfeldt-Jakob disease; subacute sclerosing panencephalitis, Hallerrorden-Spatz disease; boxer dementia; neurological trauma (such as spinal cord injury, brain injury, concussion, repetitive concussion); pain; inflammatory pain; autism; depression and major depression; stroke; cognitive impairment; epilepsy, etc. Such methods may optionally include administering an effective amount of the composition or pharmaceutical composition comprising at least one TNF antibody or specific portions or variants to the cells, tissues, organs, animals or patients in need of such modulation, treatment or therapy. See, for example, the Merck Manual, 16th edition, Merck & Company, Rahway, N.J. (1992).

The IL-6-related wound, trauma or tissue damage or related chronic conditions include, but are not limited to, at least one of the following diseases: physical injury or trauma related to oral surgery including periodontal surgery, tooth extraction, endodontic treatment, insertion of dental implants, application and use of dental restorations; or wherein the wound is selected from aseptic wounds, contusions, cuts, lacerations, non-penetrating wounds, open wounds, penetrating wounds, piercing wounds, puncture wounds, poisoned wounds, infarct formation and subcutaneous wounds; or wherein the wounds are selected from ischemic ulcers, ischemic ulcers, fistulas, severe bites, thermal burns, and donor site wounds; or wherein the wounds are aphthous wounds, traumatic wounds, or herpes-related wounds.

As used in the present invention, the term “Fab fragment” consists of one light chain and a CH1 and a variable region of one heavy chain. The heavy chain of the Fab molecule cannot form a disulfide bond with another heavy chain molecule.

As used in the present invention, the term “Fc” region contains two heavy chain fragments comprising a CH1 and a CH2 domain of an antibody. The two heavy chain fragments are associated together by two or more disulfide bonds and by hydrophobic interactions of CH3 domains.

As used in the present invention, the term “Fab′ fragment” contains one light chain and a portion of one heavy chain (which contains a VH domain and a CH1 domain and also a region between the CH1 and CH2 domains), so that an interchain disulfide bond can be formed between the two heavy chains of two Fab′ fragments to form a F(ab′)₂ molecule.

As used in the present invention, the term “F(ab′)₂ fragment” contains two light chains and two heavy chains containing the constant region between the CH1 and CH2 domains, so as to form interchain disulfide bonds between the two heavy chains. The F(ab′)₂ fragment thus consists of two Fab′ fragments associated together by the disulfide bonds between the two heavy chains.

As used in the present invention, the term “Fv region” includes variable regions derived from heavy and light chains, but lacks constant regions.

As used in the present invention, the term “Fd” fragment means an antibody fragment composed of VH and CH1 domains (Ward et al., Nature 341:544-546 (1989)).

As used in the present invention, the term “dAb” fragment (Ward et al., Nature 341:544-546 (1989)) consists of a VH domain.

As used in the present invention, the term “Fab′-SH” is the designation for Fab′ herein, in which one or more cysteine residues of the constant domain carry a free thiol group.

As used in the present invention, the term “Fab/c” fragment is an intermediate product of antibody cleavage formed by pepsin digestion of immunoglobulins. It has the advantages of both Fab and Fc regions, that is, it has strong diffusion ability and slow metabolism in the body, and can maintain high affinity (Liu Jianjun, “Journal of Cellular and Molecular Immunology”, 1989(4):29-29).

As used in the present invention, the term “single chain antibody” is an Fv molecule in which the variable regions of the heavy chain and the light chain are connected by a flexible linker to form a single polypeptide chain (which forms an antigen binding region) (see, for example, Bird et al., Science. 242:423-426 (1988) and Huston et al., Proc. Natl. Acad. Sci. USA. 90:5879-5883 (1988)). Single chain antibodies are described in detail in International Patent Application Publication No. WO 88/01649 and U.S. Pat. Nos. 4,946,778 and 5,260,203 (the disclosures of said applications are incorporated by reference).

As used in the present invention, the term “domain antibody” is an immunofunctional immunoglobulin fragment containing only a heavy chain variable region or a light chain variable region. In some cases, two or more VH regions are covalently linked by a peptide linker to generate a multivalent domain antibody (especially a bivalent domain antibody). The two VH regions of a bivalent domain antibody can target the same antigen or different antigens.

As used in the present invention, the term “bivalent antigen-binding protein” or “bivalent antibody” includes two antigen-binding sites. In some cases, the two binding sites have the same antigen specificity. The bivalent antibody can be bispecific.

As used in the present invention, the term “multispecific antigen-binding protein” or “multispecific antibody” is an antigen-binding protein or antibody that targets more than one antigen or epitope.

As used in the present invention, the term “bispecific”, “dual specific” or “bifunctional” antigen-binding protein or antibody is a hybrid antigen-binding protein or antibody with two different antigen-binding sites. A bispecific antibody is a multispecific antigen-binding protein or multispecific antibody, and can be produced by a variety of methods, including, but not limited to, the fusion of hybridomas or the linking of Fab′ fragments. See, for example, Songsivilai and Lachmann, 1990, Clin. Exp. Immunol. 79: 315-321; Kostelny et al., 1992, J. Immunol. 148: 1547-1553. The two binding sites of a bispecific antigen-binding protein or antibody will bind to two different epitopes that are present on the same protein target or different protein targets.

The “humanized” form of a non-human (e.g., murine) antibody is a chimeric antibody that contains a minimal sequence derived from non-human immunoglobulins. Most of the humanized antibodies are human immunoglobulins, in which the hypervariable region residues of the receptor antibody are replaced by the residues of the hypervariable region of non-human species (for example, mouse, rat, rabbit or non-human primate) with the required specificity, affinity and ability. In some cases, the Fv framework residues of the human immunoglobulin are replaced with corresponding non-human residues. In addition, humanized antibodies may contain residues that are not present in the receptor antibody or the donor antibody. These modifications are made to further improve the property of antibody.

When referring to ligand/receptor, antibody/antigen or other binding pairs, “specific” binding refers to determining whether there is binding reaction of the protein, such as IL-6, in a heterogeneous population of proteins and/or other biological agents. Therefore, under the specified conditions, a specific ligand/antigen binds to a specific receptor/antibody, and does not bind to other proteins present in the sample in a significant amount.

As used in the present invention, the term “humanized antibody” refers to an antibody or antibody fragment obtained by replacing all or part of the CDR region of a human immunoglobulin (receptor antibody) with a CDR region of a non-human antibody (donor antibody), wherein the donor antibody can be a non-human (eg, mouse, rat, or rabbit) antibody with the desired specificity, affinity or reactivity. In addition, some amino acid residues in the framework region (FR) of the receptor antibody can also be replaced by corresponding amino acid residues of non-human antibodies, or by amino acid residues of other antibodies, to further improve or optimize the antibody property. For more details about humanized antibodies, see, for example, Jones et al., Nature, 321:522 525 (1986); Reichmann et al., Nature, 332:323 329 (1988); Presta, Curr. Op Struct. Biol., 2:593 596 (1992); and Clark, Immunol. Today 21: 397 402 (2000).

As used herein, the terms “similarity” or “sequence similarity” and “identity” refer to the relationship between the sequences of two or more protein or polypeptide molecules, as determined by alignment and comparison of sequences. “Percent identity” means the percentage of identical residues between the molecules being compared, and can be calculated based on the size of the smallest molecule to be compared. In order to perform these calculations, a specific mathematical model or computer program (ie, “algorithm”) must be used to resolve the gaps (if any) in the alignment. When used in polypeptides, the term “substantially identical” means that two peptide sequences (when being optimally aligned by using for example the programs GAP or BESTFIT, using the default gap weights provided by the program) share at least 70%, 75%, or 80% sequence identity, at least 90% or 95% sequence identity, and at least 97%, 98%, or 99% sequence identity. In some cases, residues that are not identical differ by conservative amino acid substitutions. A “conservative amino acid substitution” is a substitution in which an amino acid residue is replaced by another amino acid residue having a side chain R group with similar chemical properties (e.g., charge or hydrophobicity). Generally, conservative amino acid substitutions will not substantially change the functional properties of the protein. When two or more amino acid sequences differ from each other by conservative substitutions, the percent sequence identity can be adjusted upwards to correct for the conservative nature of the substitutions. Means for making this adjustment are well known to those skilled in the art. See, for example, Pearson, Methods Mol. Biol. 243:307-31 (1994). Examples of amino acid groups having side chains with similar chemical properties include: 1) aliphatic side chains: glycine, alanine, valanine, leucine, and isoleucine; 2) aliphatic hydroxyl side chains: serine and threonine; 3) amide-containing side chain: asparagine and glutamine; 4) aromatic side chain: phenylalanine, tyrosine and tryptophan; 5) basic side chain: lysine, arginine and histidine; 6) acidic side chain: aspartic acid and glutamic acid; and 7) sulfur-containing side chain: cysteine and methionine. The conservative amino acid substitution groups are valine-leucine-isoleucine, phenylalanine-tyrosine, lysine-arginine, alanine-valine, glutamic acid-aspartate acid and asparagine-glutamine.

### BRIEF DESCRIPTION OF THE DRAWINGS

FIG. 1 shows the SDS-PAGE image of recombinant human IL-6.

FIG. 2 shows that hybridoma cell line 140-4 reduces the phosphorylation level of downstream signal protein p-Stat3 (Tyr705).

FIG. 3 shows the affinity of humanized monoclonal antibodies HZ-0408a, HZ-0408b, HZ-0408c and HZ-0408d to human IL-6.

FIG. 4 shows that humanized monoclonal antibodies HZ-0408a, HZ-0408b, HZ-0408c and HZ-0408d inhibit the binding of IL-6 and IL-6R.

FIG. 5 shows that humanized monoclonal antibodies HZ-0408a, HZ-0408b, HZ-0408c, and HZ-0408d inhibit the phosphorylation of p-Stat3 (Tyr705) stimulated by IL-6.

FIG. 6 shows that humanized antibody inhibits rhIL-6-stimulated SAA secretion of HepG2 cells.

FIG. 7 shows the cross-reaction results of humanized antibodies to various IL-6. FIG. 7A shows the cross-reactions of humanized antibodies to rhesus IL-6. FIG. 7B shows the cross-reactions of humanized antibodies to mouse IL-6. FIG. 7C shows the cross-reactions of humanized antibodies to rat IL-6.

### DETAILED DESCRIPTION OF THE INVENTION

The present invention will be described in detail below by specific examples. It will be understood by those skilled in the art that the following examples are for illustrative purposes.

The spirit and protection scope of the present invention are defined by the appended claims.

### Example 1. Preparation of Human IL-6

A prokaryotic expression vector for human IL-6 was constructed and transformed into E. coli BL21 (DE3), and then IL-6 expression was induced by IPTG. The inclusion-body protein is denatured and renatured, and purified by nickel column to prepare human IL-6 proteins for mouse immunization, clone screening and functional identification.

(1) Construction of Prokaryotic Expression Vector for Human IL-6

Firstly, the target sequence of human IL-6 was synthesized by genetic engineering (synthesized by Nanjing GenScript). The sequence starts from Val at position 30 of natural human IL-6 to Met at position 212 with a total of 183 amino acids (SEQ ID NO: 1). The C-terminal was added with 6 His which can bind the nickel chloride in the nickel column to facilitate the purification by ion affinity chromatography, and two enzyme cleavage sites, NdeI and XhoI, were added at both ends. The synthesized human IL-6 and the expression vector pET22b(+) (provided by Nanjing GenScript) were both digested with NdeI and XhoI; the human IL-6 fragment of interest and the expression vector fragment were recovered, ligated and transformed, The positive clones were identified by PCR and enzyme digestion, and finally the expression vector was verified to be correct by sequencing, and was named as pET22b-rhIL-6-His. The plasmid was extracted for transformation by using plasmid extraction kit.

(2) IPTG-Induced Expression and Denaturation and Renaturation of Inclusion Bodies pET22b-hIL-6-His was transformed into Escherichia coli BL21 (DE3), a single clone was picked and cultured in 5 ml LB broth containing ampicillin (50 μg/mL) at 37° C. overnight. The overnight bacteria were inoculated at 1:100 into the corresponding fresh medium and cultured at 37° C. When the bacteria grew to an OD600 of 0.6, 0.1 mM IPTG (Amresco, 0487) was added and the expression was induced at 37° C. for 6 hours.

After IPTG-induced expression, the inclusion bodies were formed, which were insoluble. The method of washing and dissolving the inclusion bodies was as follows: after resuspending the bacteria in an inclusion-body ultrasonic buffer (20 mmol/L Tris-HCl pH8.0, 0.5 mol/L NaCl, 1 mmol/L EDTA) for ultrasonic disruption, the inclusion body pellet was washed twice with an inclusion body washing buffer (20 mmol/L Tris-HCl pH 8.0, 0.5 mol/L NaCl, 2 mol/L urea, 2% Triton). Subsequently, the inclusion bodies were dissolved in inclusion body dissolution buffer (8M Urea, 25 mM Tris, 150 mM NaCl, 25 mM DTT, pH 8.0), stirred at room temperature for 5-6 hours or overnight, and centrifuged to collect the supernatant.

The concentration of the dissolved inclusion body protein was adjusted to 1 mg/ml; 1 ml of dissolved inclusion body protein was taken and put into a dialysis bag, and then placed in 140 ml external dialysis solution (6M urea, 200 mM arginine, 25 mM Tris (pH8.0), 150 mM NaCl, 2 mM reduced glutathione (GSH), 1 mM oxidized glutathione (GSSG)), standing for dialysis overnight at 4° C. 50 ml of the above-mentioned external dialysis solution was removed and 50 ml of Diluent 1 (600 mM arginine, 25 mM Tris (pH8.0), 150 mM NaCl, 2 mM GSH, 1 mM GSSG) was added. At this time, the urea concentration of the external dialysis solution was 4M. Dialysis was performed at 4° C. for 6 hours. 75 ml of the external dialysis solution was removed and 75 ml of Diluent 1 was added; the final concentration of urea was 2M. Dialysis was performed at 4° C. for 6 hours. The dialysis solution was replaced with 200 ml external dialysis solution B (400 mM arginine, 25 mM Tris, 150 mM NaCl, 2 mM GSH, 1 mM GSSG); dialysis was performed overnight at 4° C. 100 ml of the external dialysis solution was removed and 100 ml of Diluent 2 (25 mM Tris, 150 mM NaCl) was added; dialysis was performed at 4° C. for 6 hours. 100 ml of the external dialysis solution was removed and 100 ml of Diluent 2 was added; dialysis was performed at 4° C. for 6 hours. Then the solution was changed to 1 L of fresh diluent 2 and dialysis was performed overnight.

(3) Purification of rhIL-6-His

After Ni-NTA sepharose 6 Fast Flow (GE Health Care, 17-5318-02), the protein was equilibrated in 25 mM equilibration solution (Tris-HCl (pH8.0), 150 mM NaCl). Then the hIL-6-His renatured protein was loaded on the column and the column was wash with a washing solution (25 mM Tris-HCl (pH8.0), 150 mM NaCl, 50 mM imidazole). Finally, the protein on the column was eluted by elution solution (25 mM Tris-HCl (pH8.0), 150 mM NaCl, 300 mM imidazole).

(4) Identification of rhIL-6-His

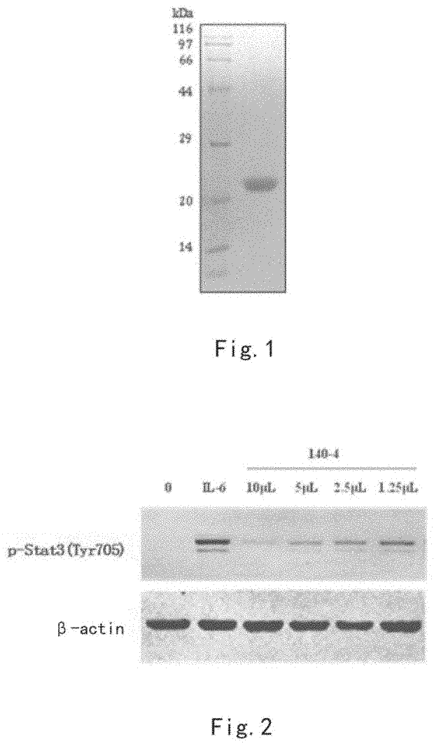

The protein content was measured by BCA method (Applygen, P1151-1), and the concentration can reach more than 1 mg/ml. The protein purity was detected by SDS-PAGE method (see FIG. 1). The purity can reach more than 95%.

### Example 2. Mouse Immunization and Determination of Antibody Titer in Serum

KM mice were immunized by RhIL-6-His as an antigen. The antigen for immunization (rhIL-6-His) was obtained in Example 1, and KM mice were purchased from Beijing Vital River Laboratory Animal Technology Co., Ltd. The route of immunization was subcutaneous multi-point injection, and the immunization dose was 100 μg/200 μl/mouse. A total of 5 mice were immunized. For the first immunization, 100 μg rhIL-6-His was mixed with 100 μl Freund's complete adjuvant (Sigma, F5881); and for the second and third immunizations, 100 μg rhIL-6-His was mixed with 100 μl Freund's incomplete adjuvant (Sigma, F5881); for the fourth (boost) immunization, 100 μg rhIL-6-His was used without any adjuvant. The time for the four immunizations was 0, 14, 28, and 39 days respectively.

After the third immunization (day 35) of 5 mice, blood was collected from the eyeballs, and the titer of anti-human IL-6 antibody in the serum of the immunized mice was determined by ELISA. First, the rhIL-6-Hiss in Example 1 was diluted to 1 μg/ml by a coating buffer (NaHCO₃ 8.4 g/L, pH 9.6), added to a 96-well ELISA plate (Corning, Acton, Mass.) at 100 μl/well, 4° C. overnight. The next day, the plate was washed with PBST (0.5% c) for 3 times; a blocking solution (3% BSA in 1×PBS) was added; the antibody was blocked for 1 hour at room temperature. The plate was washed for 3 times; the above mouse serum was 4-fold diluted with 0.5% BSA/PBS starting from 1:1000, with a blank well of 0.5% BSA/PBS; 100 μl/well was added to a ELISA plate, incubated at room temperature for 2 hours; the plate was washed for 3 times; goat anti-mouse IgG (H+L)-HRP (ProteinTech, SA00001-1) was added at a final concentration of 1 μg/ml, and incubated for 1 hour at room temperature. The plate was washed for 3 times; color development was performed for 10-20 minutes at room temperature by adding TMB (Zuman Bio, ZD311) color-developing solution, and was stopped by adding a stop solution; the absorbance at 450 nm wavelength was read on a microplate reader (BioTek, ELx808). A clone whose OD value was 2 times greater than that of the blank well was defined as a positive clone. At the highest dilution of the serum, a higher OD value indicated a stronger immunoreactivity to human IL-6.

After the third immunization, the serum titer of mouse No. 4 was 1:512000, and that of the remaining mice reached 1:128000.

### Example 3. Preparation of Hybridoma

After the last booster immunization (day 42), the spleen of mouse No. 4 with the highest serum titer was taken, ground in normal saline, and then a suspension rich in B cells was taken; the B cells and the myeloma cells SP2/0 were fused by a fusion agent PEG (Sigma, P7181). The fused cells were distributed into 15 96-well plates and cultured in a 20% fetal bovine serum RPMI-1640 complete medium (Thermo, 31800089) containing HAT (Sigma, H0262) in 5% CO₂ at 37° C. for a week.

### Example 4. Screening of Positive Hybridoma Clones

(1) Enzyme-Linked Immunosorbent Assay (ELISA) was Performed to Screen Positive Hybridoma Clones with Strong Binding Activity to the Antigen hIL-6

The first round of screening for positive cell lines was performed by an ELISA plate coated with recombinant protein human rhIL-6-His. After the first round of screening, 331 positive hybridoma monoclones with OD value>1.0 were selected.

The selected 331 positive cell lines was subjected to a second round of ELISA positive cell line screening by rhIL-6-His; a cross-screening of His tag protein and an exclusion screening of IgM subtypes were performed by conventional methods. A non-IgM cell line that was positive for rhIL-6-His and negative for His-tag protein was selected. In the end, 250 hybridoma cell lines were selected.

(2) Screening of Positive Hybridoma Clones with Strong Binding Activity to Natural IL-6 by Enzyme-Linked Immunosorbent Assay (ELISA)

The rhIL-6-His antigen was diluted with PBS (pH=8.6) to 2 μg/ml, added to a microtiter plate at 100 μl/well, coated overnight at 4° C., and blocked at 37° C. for 1 hour by adding 3% BSA after washing the plate. After washing the plate, 50 μl/well of LPS (10 ug/mL) stimulated conditioned medium and 50 μl/well of culture supernatant of hybridomas obtained by the above steps were added, and then incubated at 37° C. for 1 hour. After washing the plate with PBST, the goat anti-mouse IgG antibody (ProteinTech, SA00001-1) was added. After washing the plate with PBST, the TMB color developing solution (Zuman Bio, ZD311) was added and incubated at 37° C. for 15 minutes. The absorbance at 450 nm wavelength was read on the microplate reader (BioTek, ELx808), and 50 hybridomas with large differences in OD values were selected for subsequent screening.

(3) Screening of Positive Hybridomas Clones with Strong Neutralizing Activity for rhIL-6 by Western Blot

Different volumes of ascites from the above positive hybridoma clones and 25 ng/ml rhIL-6-His were mixed in a RPMI-1640 medium containing 10% fetal bovine serum and incubated at 37° C. for 2 hours. Then the mixture was added to DLD-1 cell ATCC® CCL-221™, incubated at 37° C. for 30 minutes, washed 3 times with PBS; RIPA lysis buffer was added to lyse the cells and the protein was collected. After SDS-PAGE electrophoresis of the protein sample, the phosphorylation level of p-STST3 (Tyr705) (Cell Signaling, 52075) was detected by Western Blot, and β-actin was used as a control.

It can be seen from FIG. 2 that clone 140-4 can block the binding of IL-6 to the receptor IL-6R, inhibit the signal transduction of gp130 and reduce the phosphorylation level of its downstream signal protein p-Stat3 (Tyr705).

The hybridoma cell line 140-4 was deposited in the China General Microbiological Culture Collection Center (CGMCC, No. 3 Beichen West Road, Chaoyang District, Beijing, the Institute of Microbiology, Chinese Academy of Sciences) on Sep. 26, 2018 with a deposit number CGMCC No. 16389.

### Example 5. Obtaining of an Murine Monoclonal Antibody

The hybridoma clone 140-4 was cultured to a total number of 10⁷ cells, and the cells were collected by centrifugation at 1000 rpm for 10 minutes, and the total RNA was extracted with Trizol kit (CWBio, CW0580S). After synthesizing a first-strand cDNA (CWBio, CW0744M) using the RNA as a template, the first-strand cDNA was used as a subsequent template to amplify the variable region DNA sequence corresponding to the hybridoma cell. The primer sequences used in the amplification reaction were complementary to the first framework region of the antibody variable region and the constant region. See (Larrick, J W, et al. (1990), Scand. J. Immunol., 32: 121-128 and Coloma, J. J. et al., (1991) BioTechniques, 11, 152-156). The Taq enzyme was used (NEB, M0491S).

SEQ ID NO: 2, the amino acid sequence of the murine monoclonal antibody heavy chain;

SEQ ID NO: 3, the amino acid sequence of the murine monoclonal antibody light chain;

SEQ ID NO: 4, the amino acid sequence of the heavy chain variable region of the murine monoclonal antibody;

SEQ ID NO: 5, the amino acid sequence of the light chain variable region of the murine monoclonal antibody;

SEQ ID NO: 6, CDR1 sequence of the murine monoclonal antibody heavy chain;

SEQ ID NO: 7, CDR2 sequence of the murine monoclonal antibody heavy chain;

SEQ ID NO:8, CDR3 sequence of the murine monoclonal antibody heavy chain;

SEQ ID NO: 9, CDR1 sequence of the murine monoclonal antibody light chain;

SEQ ID NO: 10, CDR2 sequence of the murine monoclonal antibody light chain;

SEQ ID NO: 11, CDR3 sequence of the murine monoclonal antibody light chain;

SEQ ID NO:12, FR1 sequence of the murine monoclonal antibody heavy chain;

SEQ ID NO:13, FR2 sequence of the murine monoclonal antibody heavy chain;

SEQ ID NO: 14, FR3 sequence of the murine monoclonal antibody heavy chain;

SEQ ID NO: 15, FR4 sequence of the murine monoclonal antibody heavy chain;

SEQ ID NO: 16, FR1 sequence of the murine monoclonal antibody light chain;

SEQ ID NO: 17, FR2 sequence of the murine monoclonal antibody light chain;

SEQ ID NO: 18, FR3 sequence of the murine monoclonal antibody light chain;

SEQ ID NO: 19, FR4 sequence of the murine monoclonal antibody light chain;

SEQ ID NO: 20, the nucleotide sequence of the murine monoclonal antibody heavy chain;

SEQ ID NO: 21, the nucleotide sequence of the murine monoclonal antibody light chain;

SEQ ID NO: 22, the nucleotide sequence of the heavy chain variable region of the murine monoclonal antibody;

SEQ ID NO: 23, the nucleotide sequence of the light chain variable region of the murine monoclonal antibody.

### Example 6. Humanized Modification and Performance Verification of the Murine Antibody

According to the variable region sequence of the antibody secreted by the above obtained hybridoma cell 140-4, humanized modification was performed to obtain the specific sequences as follows:

SEQ ID NO: 24, amino acid sequence of the humanized light chain L1;

SEQ ID NO: 25, nucleotide sequence of the humanized light chain L1;

SEQ ID NO: 26, amino acid sequence of the humanized light chain L1 variable region;

SEQ ID NO: 27, nucleotide sequence of the humanized light chain L1 variable region;

SEQ ID NO: 28, FR1 amino acid sequence of the humanized light chain L1 variable region;

SEQ ID NO: 29, FR2 amino acid sequence of the humanized light chain L1 variable region;

SEQ ID NO: 30, FR3 amino acid sequence of the humanized light chain L1 variable region;

SEQ ID NO: 31, FR4 amino acid sequence of the humanized light chain L1 variable region;

SEQ ID NO: 32, amino acid sequence of the humanized light chain L2;

SEQ ID NO: 33, nucleotide sequence of the humanized light chain L2;

SEQ ID NO: 34, amino acid sequence of the humanized light chain L2 variable region;

SEQ ID NO: 35, nucleotide sequence of the humanized light chain L2 variable region;

SEQ ID NO: 36, FR1 amino acid sequence of the humanized light chain L2 variable region;

SEQ ID NO: 37, FR2 amino acid sequence of the humanized light chain L2 variable region;

SEQ ID NO: 38, FR3 amino acid sequence of the humanized light chain L2 variable region;

SEQ ID NO: 39, FR4 amino acid sequence of the humanized light chain L2 variable region;

SEQ ID NO: 40, amino acid sequence of the humanized light chain L3;

SEQ ID NO: 41, nucleotide sequence of the humanized light chain L3;

SEQ ID NO: 42, amino acid sequence of the humanized light chain L3 variable region;

SEQ ID NO: 43, nucleotide sequence of the humanized light chain L3 variable region;

SEQ ID NO: 44, FR1 amino acid sequence of the humanized light chain L3 variable region;

SEQ ID NO: 45, FR2 amino acid sequence of the humanized light chain L3 variable region;

SEQ ID NO: 46, FR3 amino acid sequence of the humanized light chain L3 variable region;

SEQ ID NO: 47, FR4 amino acid sequence of the humanized light chain L3 variable region;

SEQ ID NO: 48, amino acid sequence of the humanized heavy chain H2;

SEQ ID NO: 49, nucleotide sequence of the humanized heavy chain H2;

SEQ ID NO: 50, amino acid sequence of the humanized heavy chain H2 variable region;

SEQ ID NO: 51, nucleotide sequence of the humanized heavy chain H2 variable region;

SEQ ID NO: 52, FR1 amino acid sequence of the humanized heavy chain H2 variable region;

SEQ ID NO: 53, FR2 amino acid sequence of the humanized heavy chain H2 variable region;

SEQ ID NO: 54, FR3 amino acid sequence of the humanized heavy chain H2 variable region;

SEQ ID NO: 55, FR4 amino acid sequence of the humanized heavy chain H2 variable region;

SEQ ID NO: 56, amino acid sequence of the humanized heavy chain H3;

SEQ ID NO: 57, nucleotide sequence of the humanized heavy chain H3;

SEQ ID NO: 58, amino acid sequence of the humanized heavy chain H3 variable region;

SEQ ID NO: 59, nucleotide sequence of the humanized heavy chain H3 variable region;

SEQ ID NO: 60, FR1 amino acid sequence of the humanized heavy chain H3 variable region;

SEQ ID NO: 61, FR2 amino acid sequence of the humanized heavy chain H3 variable region;

SEQ ID NO: 62, FR3 amino acid sequence of the humanized heavy chain H3 variable region;

SEQ ID NO: 63, FR4 amino acid sequence of the humanized heavy chain H3 variable region;

The nucleotide sequences of the above light chain and heavy chain were digested by HindIII and ECOR I respectively, and then ligated into pCDNA3.1 (Invitrogen, V79020) plasmid to construct an expression vector. The combinations of light chain and heavy chain are as follows: L1/H2, L1/H3, L2/H3 and L3/H3.

Twenty-four hours before transfection, 293F (Kairui Biotech, Zhuhai) was diluted with 293 medium (Kairui Biotech, Zhuhai, K03252) to a density of 3.0×10⁶ cells/ml. The cells were incubated in a constant-temperature shaker at 130 rpm, 37° C., and 5% CO₂, so that the cell density (hemocytometer) on the day of transfection was 4.0-6.0×10⁶ cells/ml. To ensure the best transfection effect, cell viability (trypan blue staining method) should be greater than 97%.

Taking the transfection of 100 ml cell suspension as an example, two 15 ml sterile centrifuge tubes were prepared; in one of the two tubes, 5 ml KPM (Kairui Biotech, Zhuhai, K03125L) and 100 μg sterile plasmid DNA were added, and mixed gently by pipetting; in another tube, 5 ml KPM and 500 μl TA-293 (Kairui Biotech, Zhuhai, K20001) transfection reagent were added, and mixed gently by pipetting; all the liquid in the centrifuge tube containing the transfection reagent was transferred to the centrifuge tube containing the plasmid, and mixed gently by pipetting; after standing at room temperature for 10 minutes, the plasmid-vector complex was prepared; cells were taken from the constant-temperature shaker, added with the prepared plasmid-vector complex under shaking, and cultured back in the CO₂ constant-temperature shaker. After 24 hours of transfection, 600 μl 293 cell protein expression-enhancer (KE-293) (Kairui Biotech, Zhuhai, K30001) and transient transfection nutritional-supplement (KT-Feed 50×) (Kairui Biotech, Zhuhai, K40001) can be added to increase the expression amount of the product; the supernatant was collected about 5 days after transfection, centrifuged at 9000 rpm in a cryocentrifuge for 20 minutes, and the supernatant was collected for the next step of protein purification.

After centrifugation, the above antibody-containing 293F cell supernatant was loaded on a Protein A column (GE Healthcare Bio-Sciences, 17-5080-02) to capture the IgG1 type antibody, and then eluted by 50 mM citric acid-sodium citrate buffer (pH=3.0); the eluate (0.5 ml) was collected, added with 100 μl 1M Tris-HCL buffer (pH=) to a neutral pH, and dialyzed in phosphate buffer PBS by a 10K dialysis membrane (Generay, M1915); the protein content was determined at OD280 nm. The protein was stored at −80° C. after filtration and sterilization. Four neutralizing antibodies HZ-0408a (L1+H2), HZ-0408b (L1+H3), HZ-0408c (L2+H3) and HZ-0408d (L3+H3) were obtained.

### Example 7. Determination of the Affinity of Humanized Antibodies by Enzyme-Linked Immunosorbent Assay (ELISA)

The rhIL-6-His antigen was diluted with PBS (pH=8.6) to 1 μg/ml, added to a microtiter plate at 100 μl/well, coated overnight at 4° C., and blocked at 37° C. for 2 hour by adding 300 μl/well of 3% BSA after washing the plate 4 times with PBST. The plate was washed once more with PBST, and added with 100 μl/well of humanized antibody at different concentrations (starting at 50 ug/ml, 5-fold dilution to 0.00064 ug/ml) and Siltuximab (Janssen, HEI15015.D) (starting at 1250 ug/ml, 5-fold dilution to 0.016 ug/ml), and incubated at 37° C. for 2 hours. The plate was washed for 4 times with PBST, added with HRP (horseradish peroxidase) labeled goat anti-human IgG antibody (ProteinTech, SA00001-1), and incubated at 37° C. for 1 hour. The plate was washed for 4 times with PBST, added with 100 μl/well of TMB color-developing solution (Zuman Bio, ZD311), and incubated at 37° C. for 15 minutes; after color development, 50 μl/well of stop solution (1M sulfuric acid) was added, and the absorbance value at 450 nm wavelength was measured on the microplate reader (BioTek, ELx808).

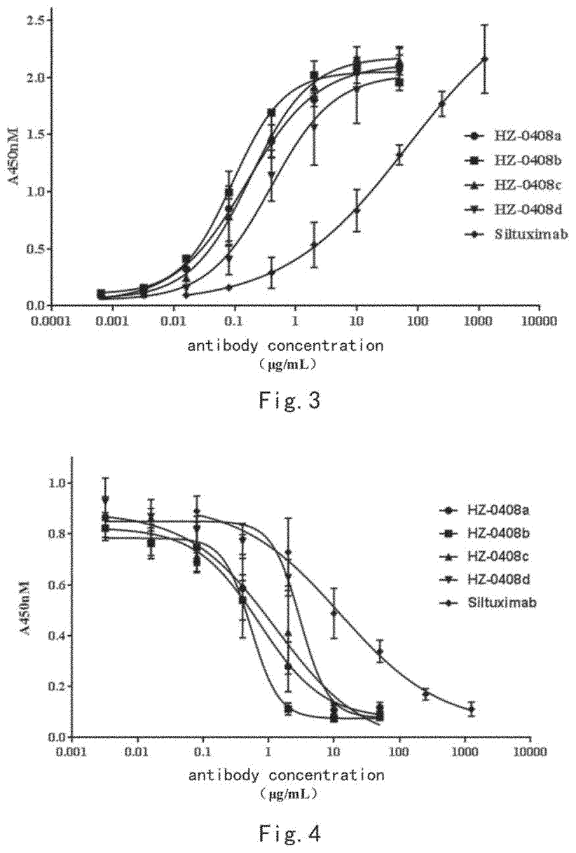

FIG. 3 shows that the four monoclonal antibodies HZ-0408a, HZ-0408b, HZ-0408c and HZ-0408d have significantly higher affinity for human IL-6 than Siltuximab.

| Antibody name | EC₅₀ (μg/mL) |
|---|---|
| HZ-0408a | 0.16 |
| HZ-0408b | 0.09 |
| HZ-0408c | 0.18 |
| HZ-0408d | 0.39 |
| Siltuximab | 67.29 |

### Example 8. Determining the Inhibition of the Binding of IL-6 and IL-6R by Humanized Antibodies by Using Enzyme-Linked Immunosorbent Assay (ELISA)

The rhIL-6R (Sino Biological, 10398-H02H) antigen was diluted with PBS (pH=8.6) to 1.5 μg/ml, added to a microtiter plate A at 100 μl/well, coated overnight at 4° C., and blocked at 37° C. for 2 hour by adding 300 μl/well of 3% BSA after washing the plate for 4 times with PBST. A microtiter plate B was added with 50 μl/well of humanized antibody at different concentrations (starting at 50 ug/ml, 5-fold dilution to 0.0032 ug/ml) and Siltuximab (starting at 1250 ug/ml, 5-fold dilution to 0.08 ug/ml) for binding to 50 μl/well of rhIL-6-His (1 μg/ml), and incubated at 37° C. for 2 hours. The plate A was washed once with PBST, added with the mixture in plate B, and incubated at 37° C. for 1 hour. The plate A was washed for 4 times with PBST, added with 100 μl/well of HRP (horseradish peroxidase) labeled anti-His antibody (ProteinTech, HRP-66005), and incubated at 37° C. for 1 hour. The plate was washed for 4 times with PBST, added with 100 μl/well of TMB color-developing solution (Zuman Bio, ZD311), and incubated at 37° C. for 15 minutes for color development; 50 μl/well of stop solution (1M sulfuric acid) was added, and the absorbance value at 450 nm wavelength was measured on the microplate reader (BioTek, ELx808).

FIG. 4 shows that the four monoclonal antibodies HZ-0408a, HZ-0408b, HZ-0408c and HZ-0408d have significantly higher inhibitory effects on the binding of IL-6 to IL-6R than Siltuximab.

| Antibody name | IC₅₀ (μg/mL) |
|---|---|
| HZ-0408a | 0.72 |
| HZ-0408b | 0.53 |
| HZ-0408c | 1.29 |
| HZ-0408d | 3.04 |
| Siltuximab | 12.46 |

### Example 9. Inhibition of STAT-3 Phosphorylation in IL-6-Stimulated DLD-1 Cells by Humanized Antibodies

The above-mentioned humanized antibodies at certain concentrations (starting at 32 ug/ml, with a 2-fold dilution to 2 ug/ml) and Siltuximab (starting at 64 ug/ml, with a 2-fold dilution to 2 ug/ml) were respectively mixed with 10 ng/ml rhIL-6-His and incubated at 37° C. for 2 hours. Then the mixture was added to DLD-1 cell ATCC® CCL-221™, incubated at 37° C. for 30 minutes, washed 3 times with PBS; RIPA lysis buffer was added to lyse the cells and the protein was collected. After SDS-PAGE electrophoresis of the protein sample, the phosphorylation level of p-STAT3 (Tyr705) (Cell Signaling, 52075) was detected by Western Blot.

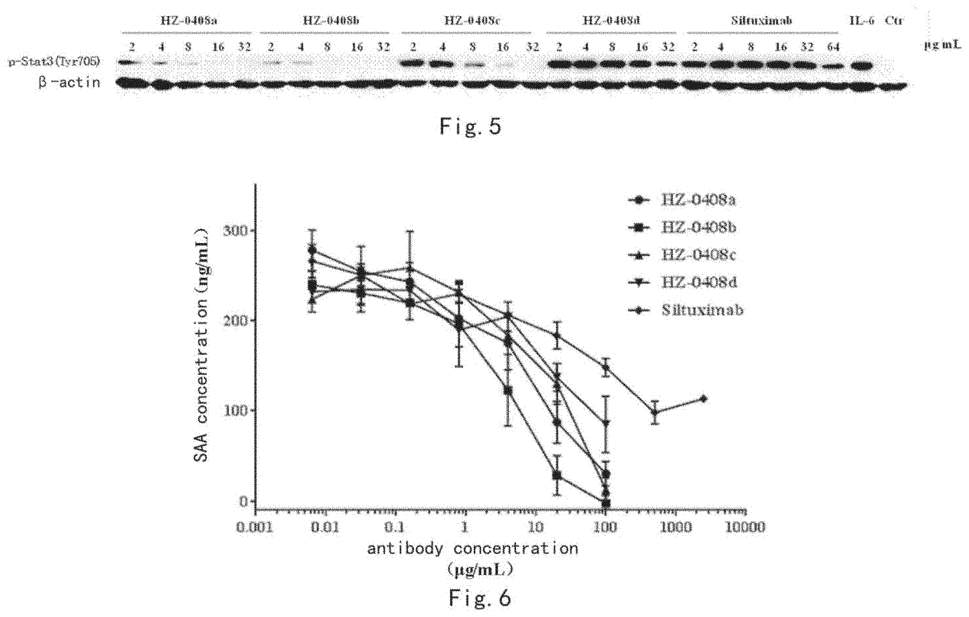

FIG. 5 shows that HZ-0408a, HZ-0408b, HZ-0408c and HZ-0408d can all inhibit the IL-6-stimulated phosphorylation of p-Stat3 (Tyr705) at lower concentrations (HZ-0408a 2 μg/ml, HZ-0408b 2 μg/ml, HZ-0408c 8 μg/ml, HZ-0408d 32 μg/ml), while Siltuximab can significantly inhibit the IL-6-stimulated phosphorylation of p-Stat3 (Tyr705) only when the concentration reaches 64 μg/ml.

### Example 10. Humanized Antibodies Inhibit SAA Secretion from rhIL-6 Stimulated HepG2 Cells

Human hepatocarcinoma cells HepG2 (Basic Medical Cell Center, Institute of Basic Medical Sciences, Chinese Academy of Medical Sciences, 3111C0001CCC000802) were inoculated into a 24-well plate at 2.25×10⁵ cells/well, and cultured in MEM NEAA medium (Thermo, 41500034) for about 24 hours. Humanized antibodies at certain concentrations (starting at 100 ug/ml, 5-fold dilution to 0.0064 ug/ml) and Siltuximab (starting at 2500 ug/ml, 5-fold dilution to 0.0064 ug/ml) were mixed with 100 ng/mL rhIL-6-His and 200 ng/mL rhIL-6R (Sino Biological, 10398-H02H), and incubated for 30 minutes; 25 ng/ml IL-10 (Sino Biological, 10139-HNAE) was added and mixed. The above mixture was added to HepG2 cells, and cultured for 48 hours; the culture supernatant was collected. ELISA kit (R&D, DY3019-05) was used to determine SAA in the supernatant.

FIG. 6 shows that HZ-0408a, HZ-0408b, HZ-0408c, HZ-0408d and Siltuximab can inhibit rhIL-6 stimulated SAA secretion in HepG2 cells in a concentration-dependent manner; wherein, 100 μg/mL of HZ-0408a, HZ-0408b, HZ-0408c, HZ-0408d and Siltuximab acted on rhIL-6 stimulated HepG2 cells, respectively, and the concentration of SAA was 30.5±9.5 ng/mL, −2.5±6.5 ng/mL, 12.5±9.5 ng/mL, 85±22 ng/mL and 148±7 ng/mL.

### Example 11. Assay of Affinity Constant of Humanized Antibody

The affinity of HZ-0408a, HZ-0408b, HZ-0408c, HZ-0408d and Siltuximab to human IL-6 was detected by ForteBio Blitz Biomolecular Interaction Analysis (ForteBio) instrument. The measured affinity constants were shown in the table below.

| Antibody name | KD | Ka(1/Ms) |
|---|---|---|
| HZ-0408a | 4.429e−9 | 1.285e5 |
| HZ-0408b | 1.075e−9 | 2.333e5 |
| HZ-0408c | 6.488e−9 | 7.433e4 |
| HZ-0408d | 2.457e−9 | 9.317e4 |
| Siltuximab | 1.438e−8 | 2.892e4 |

### Example 12. Cross-Reaction of Humanized Antibodies to Rat IL-6, Mouse IL-6 and Monkey IL-6

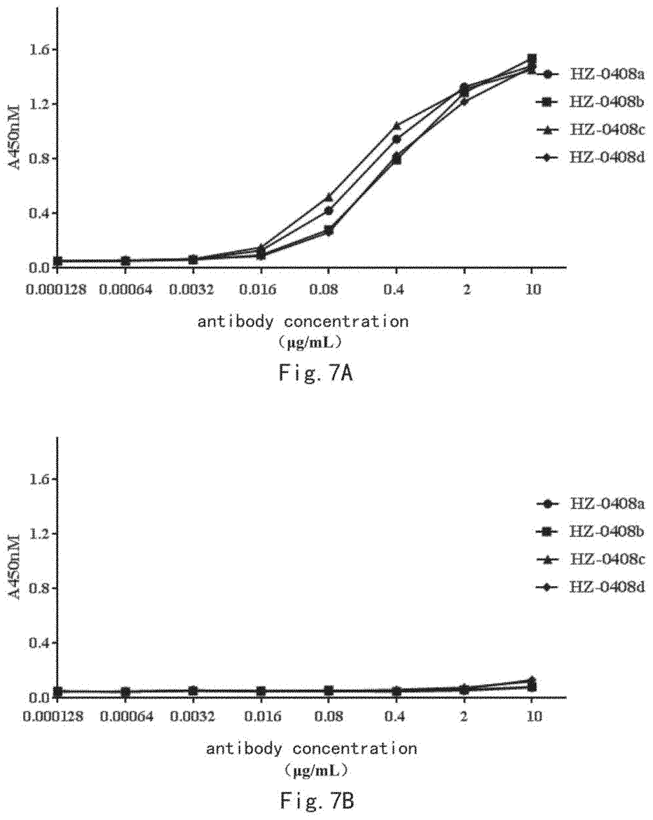

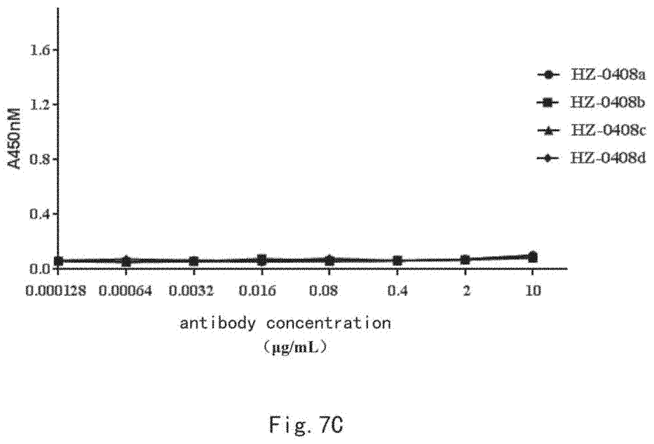

Rhesus monkey IL-6 (Sino Biological, 90197-CNAE), mouse IL-6 (Sino Biological, 50136-MNAE) and rat IL-6 (Sino Biological, 80076-RNAE) were respectively diluted to 1 μg/ml with PBS (pH=8.6), added to the microtiter plate at 100 μl/well, and coated overnight at 4° C., and blocked at 37° C. for 1 hour by adding 300 μl/well of 3% BSA after washing the plate 4 times with PBST. The plate was washed twice with PBST, and added with 100 μl/well of humanized antibodies at different concentrations (starting at 10 ug/ml, 5-fold dilution to 0.000128 ug/ml), and incubated at 37° C. for 2 hours. The plate was washed for 4 times with PBST, added with HRP (horseradish peroxidase) labeled goat anti-human IgG antibody (Proteintech, SA00001-1), and incubated at 37° C. for 1 hour. The plate was washed for 4 times with PBST, added with 100 μl/well of TMB color-developing solution (Zuman Bio, ZD311), and incubated at 37° C. for 15 minutes for color development; the absorbance value at 450 nm wavelength was measured on the microplate reader (Bio-Rad, Model 680 Micro reader). The results were shown in FIG. 7A (cross-reaction of humanized antibodies to rhesus IL-6), 7B (cross-reaction of humanized antibodies to mouse IL-6) and 7C (cross-reaction of humanized antibodies to rat IL-6).

1. An antibody or antigen-binding fragment thereof, in particular, the antibody or antigen-binding fragment thereof binds to IL6, preferably human IL6, wherein: (1) the antibody comprises:
(a) HCDR1, which comprises or consists of a sequence as shown in SEQ ID NO: 6, a sequence having at least 90%, preferably at least 91%, 92%, 93%, 94%, 95%, 96%, 97%, 98%, 99% or 100% sequence identity to SEQ ID NO: 6, or an amino acid sequence with one or more (preferably 2 or 3) conservative amino acid mutations (preferably substitutions, insertions or deletions) compared to SEQ ID NO: 6,
(b) HCDR2, which comprises or consists of a sequence as shown in SEQ ID NO: 7, a sequence having at least 90%, preferably at least 91%, 92%, 93%, 94%, 95%, 96%, 97%, 98%, 99% or 100% sequence identity with SEQ ID NO: 7, or an amino acid sequence with one or more (preferably 2 or 3) conservative amino acid mutations (preferably substitutions, insertions or deletions) compared to SEQ ID NO: 7, and
(c) HCDR3, which comprises or consists of a sequence as shown in SEQ ID NO: 8, a sequence having at least 90%, preferably at least 91%, 92%, 93%, 94%, 95%, 96%, 97%, 98%, 99% or 100% sequence identity with SEQ ID NO: 8, or an amino acid sequence with one or more (preferably 2 or 3) conservative amino acid mutations (preferably substitutions, insertions or deletions) compared to SEQ ID NO: 8,
and the antibody further comprises:
(i) LCDR1, which comprises or consists of an amino acid sequence as shown in SEQ ID NO: 9, a sequence having at least 90%, preferably at least 91%, 92%, 93%, 94%, 95%, 96%, 97%, 98%, 99% or 100% sequence identity with SEQ ID NO: 9, or an amino acid sequence with one or more (preferably 2 or 3) conservative amino acid mutations (preferably substitutions, insertions or deletions) compared to SEQ ID NO: 9,
(ii) LCDR2, which comprises or consists of an amino acid sequence as shown in SEQ ID NO: 10, a sequence having at least 90%, preferably at least 91%, 92%, 93%, 94%, 95%, 96%, 97%, 98%, 99% or 100% sequence identity with SEQ ID NO: 10, or an amino acid sequence with one or more (preferably 2 or 3) conservative amino acid mutations (preferably substitutions, insertions or deletions) compared to SEQ ID NO: 10, and
(iii) LCDR3, which comprises or consists of a sequence as shown in SEQ ID NO: 11, a sequence having at least 90%, preferably at least 91%, 92%, 93%, 94%, 95%, 96%, 97%, 98%, 99% or 100% sequence identity with SEQ ID NO: 11, or an amino acid sequence with one or more (preferably 2 or 3) conservative amino acid mutations (preferably substitutions, insertions or deletions) compared to SEQ ID NO: 11.

2. The antibody according to claim 1, wherein the antibody comprises:
(a) (i) a heavy chain variable region, which comprises or consists of the following sequence:
an amino acid sequence as shown in SEQ ID NO: 4, or
a sequence having at least 90%, preferably at least 91%, 92%, 93%, 94%, 95%, 96%, 97%, 98%, 99% or 100% sequence identity with SEQ ID NO: 4, or
an amino acid sequence with one or more (preferably 1, 2, 3, 4, 5, 6, 7, 8, 9 or 10) conservative amino acid mutations (preferably substitutions, insertions or deletions) compared to SEQ ID NO: 4, and
(ii) a light chain variable region, which comprises or consists of the following sequence:
an amino acid sequence as shown in SEQ ID NO: 5, or
a sequence having at least 90%, preferably at least 91%, 92%, 93%, 94%, 95%, 96%, 97%, 98%, 99% or 100% sequence identity with SEQ ID NO: 5, or
an amino acid sequence with one or (preferably 1, 2, 3, 4, 5, 6, 7, 8, 9 or 10) conservative amino acid mutations (preferably substitutions, insertions or deletions) compared to SEQ ID NO: 5;
(b) (i) a heavy chain variable region, which comprises or consists of the following sequence:
an amino acid sequence as shown in SEQ ID NO: 50, or
a sequence having at least 90%, preferably at least 91%, 92%, 93%, 94%, 95%, 96%, 97%, 98%, 99% or 100% sequence identity with SEQ ID NO: 50, or
an amino acid sequence with one or (preferably 1, 2, 3, 4, 5, 6, 7, 8, 9 or 10) conservative amino acid mutations (preferably substitutions, insertions or deletions) compared to SEQ ID NO: 50, and
(ii) a light chain variable region, which comprises or consists of the following sequence:
an amino acid sequence as shown in SEQ ID NO: 26, or
a sequence having at least 90%, preferably at least 91%, 92%, 93%, 94%, 95%, 96%, 97%, 98%, 99% or 100% sequence identity with SEQ ID NO: 26, or
an amino acid sequence with one or (preferably 1, 2, 3, 4, 5, 6, 7, 8, 9 or 10) conservative amino acid mutations (preferably substitutions, insertions or deletions) compared to SEQ ID NO: 26;
(c) (i) a heavy chain variable region, which comprises or consists of the following sequence:
an amino acid sequence as shown in SEQ ID NO: 58, or
a sequence having at least 90%, preferably at least 91%, 92%, 93%, 94%, 95%, 96%, 97%, 98%, 99% or 100% sequence identity with SEQ ID NO: 58, or
an amino acid sequence with one or (preferably 1, 2, 3, 4, 5, 6, 7, 8, 9 or 10) conservative amino acid mutations (preferably substitutions, insertions or deletions) compared to SEQ ID NO: 58, and
(ii) a light chain variable region, which comprises or consists of the following sequence:
an amino acid sequence as shown in SEQ ID NO: 26, or
a sequence having at least 90%, preferably at least 91%, 92%, 93%, 94%, 95%, 96%, 97%, 98%, 99% or 100% sequence identity with SEQ ID NO: 26, or
an amino acid sequence with one or (preferably 1, 2, 3, 4, 5, 6, 7, 8, 9 or 10) conservative amino acid mutations (preferably substitutions, insertions or deletions) compared to SEQ ID NO: 26;
(d) (i) a heavy chain variable region, which comprises or consists of the following sequence:
an amino acid sequence as shown in SEQ ID NO: 58, or
a sequence having at least 90%, preferably at least 91%, 92%, 93%, 94%, 95%, 96%, 97%, 98%, 99% or 100% sequence identity with SEQ ID NO: 58, or
an amino acid sequence with one or (preferably 1, 2, 3, 4, 5, 6, 7, 8, 9 or 10) conservative amino acid mutations (preferably substitutions, insertions or deletions) compared to SEQ ID NO: 58, and
(ii) a light chain variable region, which comprises or consists of the following sequence:
an amino acid sequence as shown in SEQ ID NO: 34, or
a sequence having at least 90%, preferably at least 91%, 92%, 93%, 94%, 95%, 96%, 97%, 98%, 99% or 100% sequence identity with SEQ ID NO: 34, or
an amino acid sequence with one or (preferably 1, 2, 3, 4, 5, 6, 7, 8, 9 or 10) conservative amino acid mutations (preferably substitutions, insertions or deletions) compared to SEQ ID NO: 34; or
(e) (i) a heavy chain variable region, which comprises or consists of the following sequence:
an amino acid sequence as shown in SEQ ID NO: 58, or
a sequence having at least 90%, preferably at least 91%, 92%, 93%, 94%, 95%, 96%, 97%, 98%, 99% or 100% sequence identity with SEQ ID NO: 58, or
an amino acid sequence with one or (preferably 1, 2, 3, 4, 5, 6, 7, 8, 9 or 10) conservative amino acid mutations (preferably substitutions, insertions or deletions) compared to SEQ ID NO: 58, and
(ii) a light chain variable region, which comprises or consists of the following sequence:
an amino acid sequence as shown in SEQ ID NO: 42, or
a sequence having at least 90%, preferably at least 91%, 92%, 93%, 94%, 95%, 96%, 97%, 98%, 99% or 100% sequence identity with SEQ ID NO: 42, or
an amino acid sequence with one or (preferably 1, 2, 3, 4, 42, 6, 7, 8, 9 or 10) conservative amino acid mutations (preferably substitutions, insertions or deletions) compared to SEQ ID NO: 42.

3. The antibody or antigen-binding fragment thereof according to claim 2, wherein the heavy chain variable region and the light chain variable region are respectively encoded by the following nucleotide sequences:
(a) (i) a nucleotide sequence as shown in SEQ ID NO: 22, or
a sequence having at least 90%, preferably at least 91%, 92%, 93%, 94%, 95%, 96%, 97%, 98%, 99% or 100% sequence identity with SEQ ID NO: 22, or
a nucleotide sequence with one or more (preferably 1, 2, 3, 4, 5, 6, 7, 8, 9 or 10) conservative mutations (preferably substitutions, insertions or deletions) compared to SEQ ID NO: 22, and
(ii) a nucleotide sequence as shown in SEQ ID NO: 23, or
a sequence having at least 90%, preferably at least 91%, 92%, 93%, 94%, 95%, 96%, 97%, 98%, 99% or 100% sequence identity with SEQ ID NO: 23, or
a nucleotide sequence with one or (preferably 1, 2, 3, 4, 5, 6, 7, 8, 9 or 10) conservative mutations (preferably substitutions, insertions or deletions) compared to SEQ ID NO: 23;
(b) (i) a nucleotide sequence as shown in SEQ ID NO: 51, or
a sequence having at least 90%, preferably at least 91%, 92%, 93%, 94%, 95%, 96%, 97%, 98%, 99% or 100% sequence identity with SEQ ID NO: 51, or
a nucleotide sequence with one or (preferably 1, 2, 3, 4, 5, 6, 7, 8, 9 or 10) conservative mutations (preferably substitutions, insertions or deletions) compared to SEQ ID NO: 51, and
(ii) a nucleotide sequence as shown in SEQ ID NO: 27, or
a sequence having at least 90%, preferably at least 91%, 92%, 93%, 94%, 95%, 96%, 97%, 98%, 99% or 100% sequence identity with SEQ ID NO: 27, or
a nucleotide sequence with one or (preferably 1, 2, 3, 4, 5, 6, 7, 8, 9 or 10) conservative mutations (preferably substitutions, insertions or deletions) compared to SEQ ID NO: 27;
(c) (i) a nucleotide sequence as shown in SEQ ID NO: 59, or
a sequence having at least 90%, preferably at least 91%, 92%, 93%, 94%, 95%, 96%, 97%, 98%, 99% or 100% sequence identity with SEQ ID NO: 59, or
a nucleotide sequence with one or (preferably 1, 2, 3, 4, 5, 6, 7, 8, 9 or 10) conservative mutations (preferably substitutions, insertions or deletions) compared to SEQ ID NO: 59, and
(ii) a nucleotide sequence as shown in SEQ ID NO: 27, or
a sequence having at least 90%, preferably at least 91%, 92%, 93%, 94%, 95%, 96%, 97%, 98%, 99% or 100% sequence identity with SEQ ID NO: 27, or
a nucleotide sequence with one or (preferably 1, 2, 3, 4, 5, 6, 7, 8, 9 or 10) conservative mutations (preferably substitutions, insertions or deletions) compared to SEQ ID NO: 27;
(d) (i) a nucleotide sequence as shown in SEQ ID NO: 59, or
a sequence having at least 90%, preferably at least 91%, 92%, 93%, 94%, 95%, 96%, 97%, 98%, 99% or 100% sequence identity with SEQ ID NO: 59, or
a nucleotide sequence with one or (preferably 1, 2, 3, 4, 5, 6, 7, 8, 9 or 10) conservative mutations (preferably substitutions, insertions or deletions) compared to SEQ ID NO: 59, and
(ii) a nucleotide sequence as shown in SEQ ID NO: 35, or
a sequence having at least 90%, preferably at least 91%, 92%, 93%, 94%, 95%, 96%, 97%, 98%, 99% or 100% sequence identity with SEQ ID NO: 35, or
a sequence with one or (preferably 1, 2, 3, 4, 5, 6, 7, 8, 9 or 10) conservative mutations (preferably substitutions, insertions or deletions) compared to SEQ ID NO: 35; or
(e) (i) a nucleotide sequence as shown in SEQ ID NO: 59, or
a sequence having at least 90%, preferably at least 91%, 92%, 93%, 94%, 95%, 96%, 97%, 98%, 99% or 100% sequence identity with SEQ ID NO: 59, or
a nucleotide sequence with one or (preferably 1, 2, 3, 4, 5, 6, 7, 8, 9 or 10) conservative mutations (preferably substitutions, insertions or deletions) compared to SEQ ID NO: 59, and
(ii) a nucleotide sequence as shown in SEQ ID NO: 43, or
a sequence having at least 90%, preferably at least 91%, 92%, 93%, 94%, 95%, 96%, 97%, 98%, 99% or 100% sequence identity with SEQ ID NO: 43, or
a nucleotide sequence with one or (preferably 1, 2, 3, 4, 5, 6, 7, 8, 9 or 10) conservative mutations (preferably substitutions, insertions or deletions) compared to SEQ ID NO: 43.

4. The antibody or antigen-binding fragment thereof according to claim 1, wherein the antibody further comprises framework regions FR-H1, FR-H2, FR-H3 and FR-H4 of the heavy chain variable region, and framework regions FR-L1, FR-L2, FR-L3 and FR-L4 of the light chain variable region, wherein
(a) FR-H1 comprises or consists of an amino acid sequence of SEQ ID NO: 12, or a sequence having at least 90%, preferably at least 91%, 92%, 93%, 94%, 95%, 96%, 97%, 98%, 99% or 100% sequence identity with SEQ ID NO: 12, or an amino acid sequence with one or (preferably 1, 2, 3, 4, 5, 6, 7, 8, 9 or 10) conservative amino acid mutations (preferably substitutions, insertions or deletions) compared to SEQ ID NO: 12;
FR-H2 comprises or consists of an amino acid sequence of SEQ ID NO: 13, or a sequence having at least 90%, preferably at least 91%, 92%, 93%, 94%, 95%, 96%, 97%, 98%, 99% or 100% sequence identity with SEQ ID NO: 13, or an amino acid sequence with one or (preferably 1, 2, 3, 4, 5, 6, 7, 8, 9 or 10) conservative amino acid mutations (preferably substitutions, insertions or deletions) compared to SEQ ID NO: 13;
FR-H3 comprises or consists of an amino acid sequence of SEQ ID NO: 14, or a sequence having at least 90%, preferably at least 91%, 92%, 93%, 94%, 95%, 96%, 97%, 98%, 99% or 100% sequence identity with SEQ ID NO: 14, or an amino acid sequence with one or (preferably 1, 2, 3, 4, 5, 6, 7, 8, 9 or 10) conservative amino acid mutations (preferably substitutions, insertions or deletions) compared to SEQ ID NO: 14;
FR-H4 comprises or consists of an amino acid sequence of SEQ ID NO: 15, or a sequence having at least 90%, preferably at least 91%, 92%, 93%, 94%, 95%, 96%, 97%, 98%, 99% or 100% sequence identity with SEQ ID NO: 15, or an amino acid sequence with one or (preferably 1, 2, 3, 4, 5, 6, 7, 8, 9 or 10) conservative amino acid mutations (preferably substitutions, insertions or deletions) compared to SEQ ID NO: 15; and
FR-L1 comprises or consists of an amino acid sequence of SEQ ID NO: 16, or a sequence having at least 90%, preferably at least 91%, 92%, 93%, 94%, 95%, 96%, 97%, 98%, 99% or 100% sequence identity with SEQ ID NO: 16, or an amino acid sequence with one or (preferably 1, 2, 3, 4, 5, 6, 7, 8, 9 or 10) conservative amino acid mutations (preferably substitutions, insertions or deletions) compared to SEQ ID NO: 16; FR-L2 comprises or consists of an amino acid sequence of SEQ ID NO: 17, or a sequence having at least 90%, preferably at least 91%, 92%, 93%, 94%, 95%, 96%, 97%, 98%, 99% or 100% sequence identity with SEQ ID NO: 17, or an amino acid sequence with one or (preferably 1, 2, 3, 4, 5, 6, 7, 8, 9 or 10) conservative amino acid mutations (preferably substitutions, insertions or deletions) compared to SEQ ID NO: 17; FR-L3 comprises or consists of an amino acid sequence of SEQ ID NO: 18, or a sequence having at least 90%, preferably at least 91%, 92%, 93%, 94%, 95%, 96%, 97%, 98%, 99% or 100% sequence identity with SEQ ID NO: 18, or an amino acid sequence with one or (preferably 1, 2, 3, 4, 5, 6, 7, 8, 9 or 10) conservative amino acid mutations (preferably substitutions, insertions or deletions) compared to SEQ ID NO: 18; FR-L4 comprises or consists of an amino acid sequence of SEQ ID NO: 19, or a sequence having at least 90%, preferably at least 91%, 92%, 93%, 94%, 95%, 96%, 97%, 98%, 99% or 100% sequence identity with SEQ ID NO: 19, or an amino acid sequence with one or (preferably 1, 2, 3, 4, 5, 6, 7, 8, 9 or 10) conservative amino acid mutations (preferably substitutions, insertions or deletions) compared to SEQ ID NO: 19;
(b) FR-H1 comprises or consists of an amino acid sequence of SEQ ID NO: 52, or a sequence having at least 90%, preferably at least 91%, 92%, 93%, 94%, 95%, 96%, 97%, 98%, 99% or 100% sequence identity with SEQ ID NO: 52, or an amino acid sequence with one or (preferably 1, 2, 3, 4, 5, 6, 7, 8, 9 or 10) conservative amino acid mutations (preferably substitutions, insertions or deletions) compared to SEQ ID NO: 52;
FR-H2 comprises or consists of an amino acid sequence of SEQ ID NO: 53, or a sequence having at least 90%, preferably at least 91%, 92%, 93%, 94%, 95%, 96%, 97%, 98%, 99% or 100% sequence identity with SEQ ID NO: 53, or an amino acid sequence with one or (preferably 1, 2, 3, 4, 5, 6, 7, 8, 9 or 10) conservative amino acid mutations (preferably substitutions, insertions or deletions) compared to SEQ ID NO: 53;
FR-H3 comprises or consists of an amino acid sequence of SEQ ID NO: 54, or a sequence having at least 90%, preferably at least 91%, 92%, 93%, 94%, 95%, 96%, 97%, 98%, 99% or 100% sequence identity with SEQ ID NO: 54, or an amino acid sequence with one or (preferably 1, 2, 3, 4, 5, 6, 7, 8, 9 or 10) conservative amino acid mutations (preferably substitutions, insertions or deletions) compared to SEQ ID NO: 54;
FR-H4 comprises or consists of an amino acid sequence of SEQ ID NO: 55, or a sequence having at least 90%, preferably at least 91%, 92%, 93%, 94%, 95%, 96%, 97%, 98%, 99% or 100% sequence identity with SEQ ID NO: 55, or an amino acid sequence with one or (preferably 1, 2, 3, 4, 5, 6, 7, 8, 9 or 10) conservative amino acid mutations (preferably substitutions, insertions or deletions) compared to SEQ ID NO: 55; and
FR-L1 comprises or consists of an amino acid sequence of SEQ ID NO: 28, or a sequence having at least 90%, preferably at least 91%, 92%, 93%, 94%, 95%, 96%, 97%, 98%, 99% or 100% sequence identity with SEQ ID NO: 28, or an amino acid sequence with one or (preferably 1, 2, 3, 4, 5, 6, 7, 8, 9 or 10) conservative amino acid mutations (preferably substitutions, insertions or deletions) compared to SEQ ID NO: 28;
FR-L2 comprises or consists of an amino acid sequence of SEQ ID NO: 29, or a sequence having at least 90%, preferably at least 91%, 92%, 93%, 94%, 95%, 96%, 97%, 98%, 99% or 100% sequence identity with SEQ ID NO: 29, or an amino acid sequence with one or (preferably 1, 2, 3, 4, 5, 6, 7, 8, 9 or 10) conservative amino acid mutations (preferably substitutions, insertions or deletions) compared to SEQ ID NO: 29;
FR-L3 comprises or consists of an amino acid sequence of SEQ ID NO: 30, or a sequence having at least 90%, preferably at least 91%, 92%, 93%, 94%, 95%, 96%, 97%, 98%, 99% or 100% sequence identity with SEQ ID NO: 30, or an amino acid sequence with one or (preferably 1, 2, 3, 4, 5, 6, 7, 8, 9 or 10) conservative amino acid mutations (preferably substitutions, insertions or deletions) compared to SEQ ID NO: 30;
FR-L4 comprises or consists of an amino acid sequence of SEQ ID NO: 31, or a sequence having at least 90%, preferably at least 91%, 92%, 93%, 94%, 95%, 96%, 97%, 98%, 99% or 100% sequence identity with SEQ ID NO: 31, or an amino acid sequence with one or (preferably 1, 2, 3, 4, 5, 6, 7, 8, 9 or 10) conservative amino acid mutations (preferably substitutions, insertions or deletions) compared to SEQ ID NO: 31;
(c) FR-H1 comprises or consists of an amino acid sequence of SEQ ID NO: 60, or a sequence having at least 90%, preferably at least 91%, 92%, 93%, 94%, 95%, 96%, 97%, 98%, 99% or 100% sequence identity with SEQ ID NO: 60, or an amino acid sequence with one or (preferably 1, 2, 3, 4, 5, 6, 7, 8, 9 or 10) conservative amino acid mutations (preferably substitutions, insertions or deletions) compared to SEQ ID NO: 60;
FR-H2 comprises or consists of an amino acid sequence of SEQ ID NO: 61, or a sequence having at least 90%, preferably at least 91%, 92%, 93%, 94%, 95%, 96%, 97%, 98%, 99% or 100% sequence identity with SEQ ID NO: 61, or an amino acid sequence with one or (preferably 1, 2, 3, 4, 5, 6, 7, 8, 9 or 10) conservative amino acid mutations (preferably substitutions, insertions or deletions) compared to SEQ ID NO: 61;
FR-H3 comprises or consists of an amino acid sequence of SEQ ID NO: 62, or a sequence having at least 90%, preferably at least 91%, 92%, 93%, 94%, 95%, 96%, 97%, 98%, 99% or 100% sequence identity with SEQ ID NO: 62, or an amino acid sequence with one or (preferably 1, 2, 3, 4, 5, 6, 7, 8, 9 or 10) conservative amino acid mutations (preferably substitutions, insertions or deletions) compared to SEQ ID NO: 62;
FR-H4 comprises or consists of an amino acid sequence of SEQ ID NO: 63, or a sequence having at least 90%, preferably at least 91%, 92%, 93%, 94%, 95%, 96%, 97%, 98%, 99% or 100% sequence identity with SEQ ID NO: 63, or an amino acid sequence with one or (preferably 1, 2, 3, 4, 5, 6, 7, 8, 9 or 10) conservative amino acid mutations (preferably substitutions, insertions or deletions) compared to SEQ ID NO: 63; and
FR-L1 comprises or consists of an amino acid sequence of SEQ ID NO: 28, or a sequence having at least 90%, preferably at least 91%, 92%, 93%, 94%, 95%, 96%, 97%, 98%, 99% or 100% sequence identity with SEQ ID NO: 28, or an amino acid sequence with one or (preferably 1, 2, 3, 4, 5, 6, 7, 8, 9 or 10) conservative amino acid mutations (preferably substitutions, insertions or deletions) compared to SEQ ID NO: 28;
FR-L2 comprises or consists of an amino acid sequence of SEQ ID NO: 29, or a sequence having at least 90%, preferably at least 91%, 92%, 93%, 94%, 95%, 96%, 97%, 98%, 99% or 100% sequence identity with SEQ ID NO: 29, or an amino acid sequence with one or (preferably 1, 2, 3, 4, 5, 6, 7, 8, 9 or 10) conservative amino acid mutations (preferably substitutions, insertions or deletions) compared to SEQ ID NO: 29;
FR-L3 comprises or consists of an amino acid sequence of SEQ ID NO: 30, or a sequence having at least 90%, preferably at least 91%, 92%, 93%, 94%, 95%, 96%, 97%, 98%, 99% or 100% sequence identity with SEQ ID NO: 30, or an amino acid sequence with one or (preferably 1, 2, 3, 4, 5, 6, 7, 8, 9 or 10) conservative amino acid mutations (preferably substitutions, insertions or deletions) compared to SEQ ID NO: 30;
FR-L4 comprises or consists of an amino acid sequence of SEQ ID NO: 31, or a sequence having at least 90%, preferably at least 91%, 92%, 93%, 94%, 95%, 96%, 97%, 98%, 99% or 100% sequence identity with SEQ ID NO: 31, or an amino acid sequence with one or (preferably 1, 2, 3, 4, 5, 6, 7, 8, 9 or 10) conservative amino acid mutations (preferably substitutions, insertions or deletions) compared to SEQ ID NO: 31;
(d) FR-H1 comprises or consists of an amino acid sequence of SEQ ID NO: 60, or a sequence having at least 90%, preferably at least 91%, 92%, 93%, 94%, 95%, 96%, 97%, 98%, 99% or 100% sequence identity with SEQ ID NO: 60, or an amino acid sequence with one or (preferably 1, 2, 3, 4, 5, 6, 7, 8, 9 or 10) conservative amino acid mutations (preferably substitutions, insertions or deletions) compared to SEQ ID NO: 60;
FR-H2 comprises or consists of an amino acid sequence of SEQ ID NO: 61, or a sequence having at least 90%, preferably at least 91%, 92%, 93%, 94%, 95%, 96%, 97%, 98%, 99% or 100% sequence identity with SEQ ID NO: 61, or an amino acid sequence with one or (preferably 1, 2, 3, 4, 5, 6, 7, 8, 9 or 10) conservative amino acid mutations (preferably substitutions, insertions or deletions) compared to SEQ ID NO: 61;
FR-H3 comprises or consists of an amino acid sequence of SEQ ID NO: 62, or a sequence having at least 90%, preferably at least 91%, 92%, 93%, 94%, 95%, 96%, 97%, 98%, 99% or 100% sequence identity with SEQ ID NO: 62, or an amino acid sequence with one or (preferably 1, 2, 3, 4, 5, 6, 7, 8, 9 or 10) conservative amino acid mutations (preferably substitutions, insertions or deletions) compared to SEQ ID NO: 62;
FR-H4 comprises or consists of an amino acid sequence of SEQ ID NO: 63, or a sequence having at least 90%, preferably at least 91%, 92%, 93%, 94%, 95%, 96%, 97%, 98%, 99% or 100% sequence identity with SEQ ID NO: 63, or an amino acid sequence with one or (preferably 1, 2, 3, 4, 5, 6, 7, 8, 9 or 10) conservative amino acid mutations (preferably substitutions, insertions or deletions) compared to SEQ ID NO: 63; and
FR-L1 comprises or consists of an amino acid sequence of SEQ ID NO: 36, or a sequence having at least 90%, preferably at least 91%, 92%, 93%, 94%, 95%, 96%, 97%, 98%, 99% or 100% sequence identity with SEQ ID NO: 36, or an amino acid sequence with one or (preferably 1, 2, 3, 4, 5, 6, 7, 8, 9 or 10) conservative amino acid mutations (preferably substitutions, insertions or deletions) compared to SEQ ID NO: 36;
FR-L2 comprises or consists of an amino acid sequence of SEQ ID NO: 37, or a sequence having at least 90%, preferably at least 91%, 92%, 93%, 94%, 95%, 96%, 97%, 98%, 99% or 100% sequence identity with SEQ ID NO: 37, or an amino acid sequence with one or (preferably 1, 2, 3, 4, 5, 6, 7, 8, 9 or 10) conservative amino acid mutations (preferably substitutions, insertions or deletions) compared to SEQ ID NO: 37;
FR-L3 comprises or consists of an amino acid sequence of SEQ ID NO: 38, or a sequence having at least 90%, preferably at least 91%, 92%, 93%, 94%, 95%, 96%, 97%, 98%, 99% or 100% sequence identity with SEQ ID NO: 38, or an amino acid sequence with one or (preferably 1, 2, 3, 4, 5, 6, 7, 8, 9 or 10) conservative amino acid mutations (preferably substitutions, insertions or deletions) compared to SEQ ID NO: 38;
FR-L4 comprises or consists of an amino acid sequence of SEQ ID NO: 39, or a sequence having at least 90%, preferably at least 91%, 92%, 93%, 94%, 95%, 96%, 97%, 98%, 99% or 100% sequence identity with SEQ ID NO: 39, or an amino acid sequence with one or (preferably 1, 2, 3, 4, 5, 6, 7, 8, 9 or 10) conservative amino acid mutations (preferably substitutions, insertions or deletions) compared to SEQ ID NO: 39; or
(e) FR-H1 comprises or consists of an amino acid sequence of SEQ ID NO: 60, or a sequence having at least 90%, preferably at least 91%, 92%, 93%, 94%, 95%, 96%, 97%, 98%, 99% or 100% sequence identity with SEQ ID NO: 60, or an amino acid sequence with one or (preferably 1, 2, 3, 4, 5, 6, 7, 8, 9 or 10) conservative amino acid mutations (preferably substitutions, insertions or deletions) compared to SEQ ID NO: 60;
FR-H2 comprises or consists of an amino acid sequence of SEQ ID NO: 61, or a sequence having at least 90%, preferably at least 91%, 92%, 93%, 94%, 95%, 96%, 97%, 98%, 99% or 100% sequence identity with SEQ ID NO: 61, or an amino acid sequence with one or (preferably 1, 2, 3, 4, 5, 6, 7, 8, 9 or 10) conservative amino acid mutations (preferably substitutions, insertions or deletions) compared to SEQ ID NO: 61;
FR-H3 comprises or consists of an amino acid sequence of SEQ ID NO: 62, or a sequence having at least 90%, preferably at least 91%, 92%, 93%, 94%, 95%, 96%, 97%, 98%, 99% or 100% sequence identity with SEQ ID NO: 62, or an amino acid sequence with one or (preferably 1, 2, 3, 4, 5, 6, 7, 8, 9 or 10) conservative amino acid mutations (preferably substitutions, insertions or deletions) compared to SEQ ID NO: 62;
FR-H4 comprises or consists of an amino acid sequence of SEQ ID NO: 63, or a sequence having at least 90%, preferably at least 91%, 92%, 93%, 94%, 95%, 96%, 97%, 98%, 99% or 100% sequence identity with SEQ ID NO: 63, or an amino acid sequence with one or (preferably 1, 2, 3, 4, 5, 6, 7, 8, 9 or 10) conservative amino acid mutations (preferably substitutions, insertions or deletions) compared to SEQ ID NO: 63; and
FR-L1 comprises or consists of an amino acid sequence of SEQ ID NO: 44, or a sequence having at least 90%, preferably at least 91%, 92%, 93%, 94%, 95%, 96%, 97%, 98%, 99% or 100% sequence identity with SEQ ID NO: 44, or an amino acid sequence with one or (preferably 1, 2, 3, 4, 5, 6, 7, 8, 9 or 10) conservative amino acid mutations (preferably substitutions, insertions or deletions) compared to SEQ ID NO: 44;
FR-L2 comprises or consists of an amino acid sequence of SEQ ID NO: 45, or a sequence having at least 90%, preferably at least 91%, 92%, 93%, 94%, 95%, 96%, 97%, 98%, 99% or 100% sequence identity with SEQ ID NO: 45, or an amino acid sequence with one or (preferably 1, 2, 3, 4, 5, 6, 7, 8, 9 or 10) conservative amino acid mutations (preferably substitutions, insertions or deletions) compared to SEQ ID NO: 45;
FR-L3 comprises or consists of an amino acid sequence of SEQ ID NO: 46, or a sequence having at least 90%, preferably at least 91%, 92%, 93%, 94%, 95%, 96%, 97%, 98%, 99% or 100% sequence identity with SEQ ID NO: 46, or an amino acid sequence with one or (preferably 1, 2, 3, 4, 5, 6, 7, 8, 9 or 10) conservative amino acid mutations (preferably substitutions, insertions or deletions) compared to SEQ ID NO: 46;
FR-L4 comprises or consists of an amino acid sequence of SEQ ID NO: 47, or a sequence having at least 90%, preferably at least 91%, 92%, 93%, 94%, 95%, 96%, 97%, 98%, 99% or 100% sequence identity with SEQ ID NO: 47, or an amino acid sequence with one or (preferably 1, 2, 3, 4, 5, 6, 7, 8, 9 or 10) conservative amino acid mutations (preferably substitutions, insertions or deletions) compared to SEQ ID NO: 47.

5. The antibody or antigen-binding fragment thereof according to claim 1, wherein the antibody comprises or consists of an amino acid sequence with the following heavy chain and light chain combinations:
(a) an amino acid sequence of SEQ ID NO: 2 and an amino acid sequence of SEQ ID NO: 3;
(b) an amino acid sequence of SEQ ID NO: 48 and an amino acid sequence of SEQ ID NO: 24;
(c) an amino acid sequence of SEQ ID NO: 56 and an amino acid sequence of SEQ ID NO: 24;
(d) an amino acid sequence of SEQ ID NO: 56 and an amino acid sequence of SEQ ID NO: 32; or
(e) an amino acid sequence of SEQ ID NO: 56 and an amino acid sequence of SEQ ID NO: 40, preferably, wherein the heavy chain and the light chain are respectively encoded by the following nucleotide sequences:
(i) a nucleotide sequence of SEQ ID NO: 20 and a nucleotide sequence of SEQ ID NO: 21;
(ii) a nucleotide sequence of SEQ ID NO: 49 and a nucleotide sequence of SEQ ID NO: 25;
(iii) a nucleotide sequence of SEQ ID NO: 57 and a nucleotide sequence of SEQ ID NO: 25;
(iv) a nucleotide sequence of SEQ ID NO: 57 and a nucleotide sequence of SEQ ID NO: 33; or
(v) a nucleotide sequence of SEQ ID NO: 57 and a nucleotide sequence of SEQ ID NO: 41.

6. (canceled)

7. The antibody or antigen-binding fragment thereof according to claim 1, wherein the antibody is a humanized antibody, a chimeric antibody, or a multispecific antibody (e.g., a bispecific antibody), preferably,
wherein the constant region of the antibody is humanized, preferably derived from human IgG, more preferably derived from human IgG1 or IgG4, more preferably
wherein the heavy chain constant region of the antibody adopts an Ig gamma-1 or Ig gamma-4 chain C region, preferably an Ig gamma-1 chain C region; the light chain constant region adopts an Ig kappa chain C region, more preferably an Ig kappa chain C region of the GenBank accession number ACCESSION: P01834.

8. (canceled)

9. (canceled)

10. The antibody or antigen-binding fragment thereof according to claim 1, wherein the antigen binding fragment is selected from Fab, Fab′, F(ab′)₂, Fd, Fv, dAb, Fab/c, a complementary determining region (CDR) fragment, a single-chain antibody (eg, scFv), a diabody or a domain antibody.

11-15. (canceled)

16. The antibody or antigen-binding fragment thereof according to claim 1, wherein the antibody or antigen-binding fragment thereof is in a form of an antibody conjugate, wherein the antibody conjugate comprises the antibody or antigen-binding fragment thereof according to claim 1 and a coupling moiety coupled thereto, preferably, the coupling moiety is selected from purification tags (such as His tag), cytotoxic agents, detectable labels, radioisotopes, luminescent substances, colored substances, enzymes or polyethylene glycol.

17. The antibody or antigen-binding fragment thereof according to claim 1, wherein the antibody or antigen-binding fragment thereof is in a form of a multispecific antibody, preferably a bispecific antibody or a fusion protein, which comprises the antibody or antigen-binding fragment thereof according to claim 1, as well as an antibody or antigen-binding fragment against other antigens and/or other epitopes.

18. The antibody or antigen-binding fragment thereof according to claim 1, which is contained in a kit.

19. The antibody or antigen-binding fragment thereof according to claim 1, which is contained in a pharmaceutical composition, wherein the pharmaceutical composition, optionally further comprises a pharmaceutically acceptable carrier and/or excipient.

20. The antibody or antigen-binding fragment thereof according to claim 19, wherein the pharmaceutical composition is a dosage form suitable to be administrated in a mode selected from at least one the following: parenteral administration, subcutaneous administration, intramuscular administration, intravenous administration, intraarticular administration, intrabronchial administration, intraabdominal administration, intravesicular administration, intrachondral administration, intracavitary administration, intra-bodycavitary administration, intracerebellar administration, intracerebroventricular administration, intracolonic administration, intra-neck administration, intragastric administration, intrahepatic administration, intramyocardial administration, intraosseous administration, intrapelvic administration, intrapericardial administration, intraperitoneum administration, intrapleural administration, intraprostatic administration, intrapulmonary administration, intrarectal administration, intrarenal administration, intraretinal administration, intraspinal administration, intrasynovial administration, intrathoracic administration, intrauterine administration, intravesical administration, bolus administration, vaginal administration, rectal administration, buccal administration, sublingual administration, intranasal administration, or transdermal administration.

21. The kit according to claim 18, wherein; preferably, the kit further comprises a second antibody, which specifically targets the antibody or antigen-binding fragment thereof according to claim 1 optionally, the second antibody also includes a detectable label, such as a radioisotope, a luminescent substance, a colored substance, and an enzyme.

22. (canceled)

23. (canceled)

24. A method of prevention and/or treatment and/or adjuvant treatment and/or diagnosis of diseases in a subject in need thereof comprising administering the antibody or antigen-binding fragment thereof of any one of claims 1-13 to the subject, wherein the diseases are selected from fatigue, cachexia, inflammatory diseases, autoimmune diseases, skeletal system diseases, fever, cancer, heart disease, obesity, diabetes, asthma, Alzheimer's disease, multicentric Castleman disease, multiple sclerosis and rheumatoid arthritis; IL-6 related immune-related diseases, cardiovascular diseases, infectious diseases, malignancies, neurological diseases, wound, trauma or tissue damage or related chronic conditions,
wherein preferably,
the IL-6-related immune-related diseases include but are not limited to at least one of the following:
(a) respiratory diseases selected from the group consisting of obstructive airway disease; asthma; bronchitis; acute rhinitis, allergic rhinitis, atrophic rhinitis and chronic rhinitis; membranous rhinitis; seasonal rhinitis; sarcoidosis, farmer's lung and related diseases, adult respiratory distress syndrome, allergic pneumonia, fibrotic lung and idiopathic interstitial pneumonia; neonatal chronic lung disease;
(b) bone and joint diseases selected from the group consisting of rheumatoid arthritis, childhood rheumatoid arthritis, systemic juvenile rheumatoid arthritis, juvenile chronic arthritis, seronegative spondyloarthritis (including psoriatic arthritis, ankylosing spondylitis and Wright's disease), Behcet's disease, Sjogren's syndrome, systemic sclerosis, osteoarthritis, gout, osteolysis;
(c) skin diseases selected from the group consisting of psoriasis, allergic contact dermatitis, contact dermatitis, atopic dermatitis, other eczema skin diseases, seborrheic dermatitis, lichen planus, scleroderma, pemphigus, bullous pemphigoid, epidermolysis bullosa, urticaria, rubella, xerodermas (angiodermas), vasculitis, erythema, skin eosinophilia, uveitis, alopecia areata, allergic conjunctivitis and vernal vemal conjunctivitis;
(d) gastrointestinal tract diseases
Stomach ulcer, inflammatory bowel disease, ulcerative colitis, abdominal disease, proctitis, eosinophilic gastroenteritis, mastocytosis, Crohn's disease, ulcerative colitis, antiphospholipid syndrome, food-related allergies leading to effects far away from internal organs such as migraine, rhinitis and eczema;
(e) graft rejection selected from the group consisting of graft, or graft versus host disease rejection, allograft rejection of any organ or tissue (kidney, heart, liver, pancreas, lung, bone marrow, skin, cartilage, bone, small intestine, fetal thymus, parathyroid, cornea), xenograft rejection of any organ or tissue;
(f) other tissues and systemic diseases selected from the group consisting of cachexia, systemic lupus erythematosus, skin lupus erythematosus, lupus nephritis, antiphospholipid syndrome, iridocyclitis/uveitis/optic neuritis, systemic vasculitis/Wegener's granulomatosis, Sarcoidosis, orchitis/vasectomy reversal process, allergic/atopic disease, allergic contact dermatitis, systemic inflammatory response syndrome, sepsis syndrome, gram-positive sepsis, gram-negative sepsis, culture Negative sepsis, fungal sepsis, neutropenia fever, urinary sepsis, meningococcal bacteremia, trauma/bleeding, burns, ionizing radiation exposure, acute pancreatitis, alcohol-induced hepatitis, chronic inflammatory pathology, aseptic relaxation of surgical implants, Sarcoidosis, sickle cell anemia, diabetes, nephropathy, atopic disease, hypersensitivity, hay fever, endometriosis, systemic anaphylaxis, pernicious anemia, hemolytic disease, thrombocytopenia, anti-receptor hypersensitivity, Graves' disease, Raynaud's disease, type B insulin resistant diabetes, myasthenia gravis, antibody-mediated cytotoxicity, type III hypersensitivity, POEMS syndrome (polyneuropathy, megaorganism, endocrine disease, monoclonal gammopathy and skin change syndrome), polyneuropathy, megaorganism, endocrine disease, monoclonal gammopathy, skin change syndrome, antiphospholipid symptoms, mixed connective tissue disease, primary Addison's disease, chronic active hepatitis, primary biliary cirrhosis, leukoplakia, vasculitis, post-MI cardiotomy syndrome, type IV allergies, granulomas caused by intracellular organisms, drug allergy, metabolic diseases/spontaneous disease, Wilson's disease, hemochromatosis, α-1-antitrypsin deficiency, diabetic retinopathy, Hashimoto's thyroiditis, hypothalamus-Pituitary-adrenal axis assessment, primary biliary cirrhosis, thyroiditis, encephalomyelitis, cystic fibrosis, familial hematophagocytic lymphohistiocytosis, skin medication disorders, nephrotic syndrome, nephritis, glomerulonephritis, acute renal failure, hemodialysis, uremia, poisoning, preeclampsia, okt3 therapy, anti-CD3 therapy, alopecia, cytokine therapy, chemotherapy, radiotherapy (such as including but not limited to fatigue, anemia, cachexia, etc.), chronic salicylate poisoning;
the cardiovascular diseases include but are not limited to at least one of the following diseases: cardiacstun syndrome, myocardial infarction, congestive heart failure, stroke, ischemic attack, hemorrhage, acute coronary syndrome, arteriosclerosis, atherosclerosis, restenosis, diabetes, diabetic macular edema, diabetic aterosclerotic disease, hypertension, arterial hypertension, renovascular hypertension, syncope, shock, cardiovascular system syphilis, heart failure, pulmonary (primary) heart disease, primary pulmonary hypertension, arrhythmia, atrial ectopic beats, atrial flutter, atrial fibrillation (persistent or sudden), post-perfusion syndrome, cardiopulmonary bypass inflammation, disturbed or multi-source atrial tachycardia, regular narrow QRS tachycardia, special arrhythmia, ventricular fibrillation, His bundle arrythmias, atrioventricular block, bundle branch block, myocardial ischemic disease, coronary artery disease, angina pectoris, myocardial infarction, cardiomyopathy, dilated congestive cardiomyopathy, restrictive cardiomyopathy, heart valve disease, endocarditis, pericardial disease, heart tumor, aorta and peripheral aneurysm, aortic wall dissection, aortic inflammation, abdominal aorta and its branch occlusion, peripheral vascular disease, occlusive artery disease, peripheral atherlosclerotic disease, thromboangiitis obliterans, functional peripheral artery disease, Raynaud's phenomenon and disease, hand and foot cyanosis, erythematous limb pain, venous disease, thrombophlebitis, varicose vein, arteriovenous fistula, lymphedema, lipoedema, unstable angina, reperfusion injury, post pump syndrome, ischemia-reperfusion injury;
the IL-6-related infectious diseases include but are not limited to at least one of the following diseases: acute or chronic bacterial infection, acute or chronic parasitic or infectious process, including bacterial, viral and fungal infections, HTV infection/HIV neuropathy, meningitis, Hepatitis (such as type A, type B or type C, etc.), septic arthritis, peritonitis, pneumonia, epiglottitis, E. coli 0157:h7, hemolytic uremic syndrome/thrombolytic thrombocytopenic purpura, malaria, dengue Leather hemorrhagic fever, leishmaniasis, leprosy, toxic shock syndrome, streptococcal myositis, gas gangrene, Mycobacterium tuberculosis, Mycobacterium avium intracellular parasites, Pneumocystis carinii pneumonia, pelvic inflammatory disease, orchitis/epididymitis, Legionella, Lyme disease, influenza A, Epstein-Barr virus, virus-related hemophagocytic syndrome, viral encephalitis/sterile meningitis, enterovirus 71 hand, foot and mouth disease;
the IL-6-related malignancies include but are not limited to at least one of the following diseases: leukemia, acute leukemia, acute lymphoblastic leukemia (ALL), acute lymphocytic leukemia, B-cell, T-cell or FAB ALL, Acute Myelogenous Leukemia (AML), Acute Myeloid Leukemia, Chronic Myeloid Leukemia (CML), Chronic Lymphocytic Leukemia (CLL), Hairy Cell Leukemia, Myelodysplastic Syndrome (MDS), Lymphoma, Hodgkin Disease, malignant lymphoma, non-Hodgkin's lymphoma, Burkitt's lymphoma, multiple myeloma, Kaposi's sarcoma, colorectal cancer, pancreatic cancer, nasopharyngeal carcinoma, malignant histiocytosis, paraneoplastic syndrome/malignant hypercalcemia (idiopathic) syndrome, solid tumors, bladder cancer, breast cancer, colorectal cancer, endometrial cancer, head cancer, neck cancer, hereditary non-polyposis cancer, Hodgkin's Lymphoma, liver cancer, lung cancer, non-small cell lung cancer, ovarian cancer, pancreatic cancer, prostate cancer, renal cell carcinoma, testicular cancer, adenocarcinoma, sarcoma, malignant melanoma, hemangioma, tumor metastatic disease, cancer-related bone resorption, cancer-related bone pain, etc.; inhibition of cancer metastasis; improvement of cancer cachexia;
the IL-6-related neurological diseases include but are not limited to at least one of the following diseases: neurodegenerative diseases, multiple sclerosis, migraine, AIDS dementia syndrome, demyelinating diseases, such as multiple sclerosis and acute transverse myelitis; extrapyramidal and cerebellar disorders, such as corticospinal system damage; basal ganglia disorders; hyperkinetic dyskinesias, such as Huntington's chorea and senile chorea; drug-induced dyskinesia, such as diseases induced by drugs that block CNS dopamine receptors; hypokinetic dyskinesias, such as Parkinson's disease; Progressive supranucleo Palsy; cerebellar structural damage; spinocerebellar degeneration, such as spinal ataxia, Freeh Dreich's ataxia, cerebellar cortex degeneration, multiple system degenerations (Mencel, Dejerine-Thomas, Shi-Drager and Machado-Joseph); systemic disorders (Raphthum's disease, abetalipoprotemia, ataxia, telangiectasia and mitochondrial multisystem disorders); demyelinating core disorders, such as multiple sclerosis, acute transverse myelitis; and motor unit disorders such as neuromuscular atrophy (anterior horn cell degeneration, such as amyotrophic (spinal cord) lateral sclerosis, infantile spinal muscular atrophy, and juvenile spinal muscular atrophy); Alzheimer's disease; Down syndrome in middle-aged people; diffuse Lewy body disease; Lewy body type senile dementia; Wernicke-Korsakov syndrome; chronic alcoholism; Kreuzfeldt-Jakob disease; subacute sclerosing panencephalitis, Hallerrorden-Spatz disease; boxer dementia; neurological trauma (such as spinal cord injury, brain injury, concussion, repetitive concussion); pain; inflammatory pain; autism; depression and major depression; stroke; cognitive impairment; epilepsy;
the IL-6-related wound, trauma or tissue damage or related chronic conditions include, but are not limited to, at least one of the following diseases: physical injury or trauma related to oral surgery including periodontal surgery, tooth extraction, endodontic treatment, insertion of dental implants, application and use of dental restorations; or wherein the wound is selected from aseptic wounds, contusions, cuts, lacerations, non-penetrating wounds, open wounds, penetrating wounds, piercing wounds, puncture wounds, poisoned wounds, infarct formation and subcutaneous wounds; or wherein the wounds are selected from ischemic ulcers, ischemic ulcers, fistulas, severe bites, thermal burns, and donor site wounds; or wherein the wounds are aphthous wounds, traumatic wounds, or herpes-related wounds.

25. (canceled)

26. (canceled)

27. (canceled)
